(19) United States
(12) Patent Application Publication
(10) Pub. No.: US 2009/0325976 A1
(43) Pub. Date: Dec. 31, 2009

(54) PROSTACYCLIN DERIVATIVES

(75) Inventor: Roger Tung, Lexington, MA (US)

Correspondence Address: FOLEY & LARDNER LLP, 111 HUNTINGTON AVENUE, 26TH FLOOR, BOSTON, MA, 02199-7610, US

(73) Assignee: Concert Pharmaceuticals Inc., Lexington, MA (US)

(21) Appl. No.: 12/489,425

(22) Filed: Jun. 22, 2009

Related U.S. Application Data
(63) Continuation-in-part of application No. PCT/US2007/026264, filed on Dec. 21, 2007.
Application No. PCT/US2007/026264 is a continuation-in-part of application No. 11/963,761, filed on Dec. 21, 2007, now abandoned.
(60) Provisional application No. 60/876,595, filed on Dec. 21, 2006.
(60) Provisional application No. 60/876,595, filed on Dec. 21, 2006.

(51) Int. Cl. A61K 31/19; A61P 9/12; A61K 31/496; A61K 31/506; C12N 5/00; C07C 61/28

(52) U.S. Cl. 514/252.16; 514/557; 514/269; 435/377; 562/501

(57) ABSTRACT

This invention relates to novel prostacyclin derivatives, their acceptable acid addition salts, solvates, hydrates and polymorphs thereof. The invention also provides compositions comprising a compound of this invention and the use of such compositions in methods of treating diseases and conditions beneficially treated by prostacyclin, and in particular those diseases and conditions beneficially treated by dilators of systemic and pulmonary arterial vascular beds or by platelet aggregation inhibitors.

### RELATED APPLICATIONS

This application is a continuation-in-part of U.S. application Ser. No. 11/963,761 filed Dec. 21, 2007, which claims the benefit of U.S. provisional patent application Ser. No. 60/876,595, filed Dec. 21, 2006, and is a continuation-in-part of PCT patent application no. PCT/US2007/026264, filed Dec. 21, 2007, which entered the U.S. national phase under 35 U.S.C. §371 as U.S. application Ser. No. 12/520,493, filed Jun. 19, 2009, and which claims the benefit of U.S. provisional patent application Ser. No. 60/876,595, filed Dec. 21, 2006. The contents of each of these applications are incorporated herein by reference.

### TECHNICAL FIELD OF THE INVENTION

This invention relates to novel prostacyclin derivatives, their acceptable acid addition salts, solvates, hydrates and polymorphs thereof. The invention also provides compositions comprising a compound of this invention and the use of such compositions in methods of treating diseases and conditions beneficially treated by prostacyclin, and in particular those diseases and conditions beneficially treated by dilators of systemic and pulmonary arterial vascular beds or by platelet aggregation inhibitors.

### BACKGROUND OF THE INVENTION

Iloprost is a synthetic analogue of prostacyclin PGI2 and is described in U.S. Pat. No. 4,692,464. Iloprost is known by the chemical names (E)-(3aS,4R,5R,6aS)-hexahydro-5-4-[(E)-(3S,4RS)-3-hydroxy-4-methyl-1-octen-6-ynyl]-Δ^{2(1H),Δ}-pentalenevaleric acid; and 5-[(E)-(1S, 5S, 6R, 7R)-7-hydroxy-6-[(E)-(3S, 4RS)-3-hydroxy-4-methyl-1-octen-6-inyl]-bi-cyclo[3.3.0]octan-3-ylidene)pentanoic acid.

Iloprost is known to have in vitro pharmacological effects on inhibiting platelet aggregation and platelet adhesion. It is also known to cause dilation of arterioles and venules, and has been shown to reduce vascular permeability caused by mediators such as serotonin or histamine. Iloprost has also been shown to lower pulmonary arterial pressure in animal models of pulmonary hypertension. Its ability to inhibit pulmonary vasoconstriction and reduce pulmonary vascular resistance together with its platelet anti-aggregation and antithrombotic activity are factors that favor its use in the therapeutic treatment of pulmonary arterial hypertension. Such use has been approved in the United States using an inhalable formulation of iloprost.

Despite its efficacy, and because of its short half-life, iloprost must be administered 6 to 9 times per day, not more than once every 2 hours. This high frequency of administration can lead to problems with compliance such as missed dosages, and overdosing when compensating for missed dosages. Additionally, the patient does not experience adequate therapeutic coverage during sleep. More common side effects of iloprost include abnormal lab test; back pain; blurred vision, confusion, dizziness, faintness, or lightheadedness when getting up from a lying or sitting position suddenly; chills; cough increased; coughing or spitting up blood; diarrhea; difficulty opening the mouth; feeling of warmth; fever; general feeling of discomfort or illness; headache; joint pain; lockjaw; loss of appetite; muscle aches and pains; muscle cramps; muscle spasms, especially of neck and back; nausea; redness of the face, neck, arms and occasionally, upper chest; runny nose; shivering; sore throat; sweating; trouble sleeping; sleeplessness; unable to sleep; unusual tiredness or weakness; and vomiting. These side effects may be attributable to one or more of the metabolites of iloprost and/or overdosing due to poor compliance with the high number of dosages requires on a daily basis.

Thus, despite the beneficial activities of iloprost, there is a continuing need for new and improved compounds to treat the aforementioned diseases and conditions.

### DEFINITIONS

The terms “ameliorate” and “treat” are used interchangeably and both mean decrease, suppress, attenuate, diminish, arrest, or stabilize the development or progression of a disease (e.g., a disease or disorder delineated herein).

By “disease” is meant any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of iloprost will inherently contain small amounts of deuterated isotopologues. The concentration of naturally abundant stable hydrogen and carbon isotopes, notwithstanding this variation, is small and immaterial with respect to the degree of stable isotopic substitution of compounds of this invention. See for instance See, for instance, Wada E et al, Seikagaku 1994, 66:15; Ganes L Z et al, Comp Biochem Physiol Mol Integr Physiol 1998, 119:725. In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation) at each atom designated as deuterium in said compound.

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

In other embodiment, a compound of the invention contains less than 10%, preferably less than 6%, and more preferably less than 3% of all other isotopologues combined, including a form that lacks any deuterium. In certain aspects, the compound contains less than “X” % of all other isotopologues combined, including a form that lacks any deuterium; where X is any number between 0 and 10 (e.g., 1, 0.5, 0.001), inclusive. Compositions of matter that contain greater than 10% of all other isotopologues combined are referred to herein as “mixtures” and must meet the parameters set forth below. These limits of isotopic composition and all references to isotopic composition herein, refer solely to the relative amounts of deuterium/hydrogen present in the active, free base form of the compound of Formula I or II, and do not include the isotopic composition of hydrolyzable portions of prodrugs, or of counterions.

The term “isotopologue” refers to species that differ from a specific compound of this invention only in the isotopic composition of their molecules or ions.

The term “compound” as used herein, is also intended to include salts, solvates, and hydrates thereof. The specific recitation of “salt,” “solvate,” or “hydrate,” in certain aspects of the invention described in this application shall not be interpreted as an intended omission of these forms in other aspects of the invention where the term “compound” is used without recitation of these other forms.

A salt of a compound of this invention is formed between an acid and a basic group of the compound, such as an amino functional group, or a base and an acidic group of the compound, such as a carboxyl functional group. According to another preferred embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention contain asymmetric carbon atoms at the 3 and 4 positions of the octen-6-ynyl side chain. The stereochemistry at the 3-position is S, which is the stereochemistry required for activity. As such, a compound of this invention can exist as the individual 3S,4S or 3S,4R diastereoisomers, as well a mixture of those two diastereoisomers. Accordingly, a compound of the present invention will include not only a stereoisomeric mixture, but also individual respective stereoisomers substantially free from other stereoisomers. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X” % of other stereoisomers (wherein X is a number between 0 and 100, inclusive) are present. Methods of obtaining or synthesizing diastereomers are well known in the art and may be applied as practicable to final compounds or to starting material or intermediates. Other embodiments are those wherein the compound is an isolated compound. The term “at least X % enantiomerically enriched” as used herein means that at least X % of the compound is a single enantiomeric form, wherein X is a number between 0 and 100, inclusive.

The term “stable compounds”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

The terms “lighter isotopologue” and “lighter atom isotopologue” as used herein, refer to species that differ from a specific compound of this invention in that they comprise a hydrogen atom at positions occupied by a deuterium in the specific compound.

“D” refers to deuterium.

“Stereoisomer” refers to enantiomer or diastereomer. “Tert”, “^{t}”, and “t-” refers to tertiary.

Throughout this specification, reference to “each Y” includes, independently, all “Y” groups (e.g., Y^{1a}, Y^{1b}, Y^{2a} and Y^{2b}) and reference to “each Z” includes, independently, all “Z” groups (e.g., Z^{1a} and Z^{1b}), where applicable.

### Therapeutic Compounds

The present invention provides a compound of formula I:

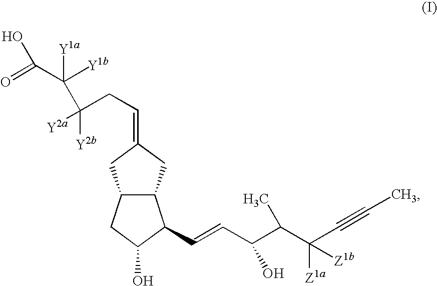

or a pharmaceutically acceptable salt thereof, wherein:

each Y is independently selected from hydrogen or deuterium;

each Z is independently selected from hydrogen, deuterium or fluorine; and

at least one Y or Z is deuterium.

In one embodiment, Y^{1a} and Y^{1b} are the same. In a more specific embodiment, Y^{1a} and Y^{1b} are simultaneously deuterium.

In another embodiment, Y^{2a} and Y^{2b} are the same. In a more specific embodiment, Y^{2a} and Y^{2b} are simultaneously deuterium.

In yet another embodiment, Z^{1a} and Z^{1b} are independently selected from deuterium or fluorine. More specifically, Z^{1a} and Z^{1b} are the same. Even more specifically, Z^{1a} and Z^{1b} are simultaneously deuterium or simultaneously fluorine. In a very specific embodiment, Z^{1a} and Z^{1b} are simultaneously deuterium.

In one particular embodiment, Y^{1a}, Y^{1b}, Y^{2a}, and Y^{2b} are simultaneously deuterium.

In another embodiment, the compound is selected from any one of the compounds set forth in the following table:

TABLE 1
Exemplary Compounds of Formula I

| Cmpd | Y^{1a} | Y^{1b} | Y^{2a} | Y^{2b} | Z^{1a} | Z^{1b} |
|---|---|---|---|---|---|---|
| 100 | D | D | H | H | H | H |
| 101 | H | H | D | D | H | H |
| 102 | D | D | D | D | H | H |
| 103 | D | D | H | H | D | D |
| 104 | H | H | D | D | D | D |
| 105 | D | D | D | D | D | D |
| 106 | D | D | H | H | F | F |
| 107 | H | H | D | D | F | F |
| 108 | D | D | D | D | F | F |
| 109 | H | H | H | H | D | D |

The present invention also provides a compound of formula II:

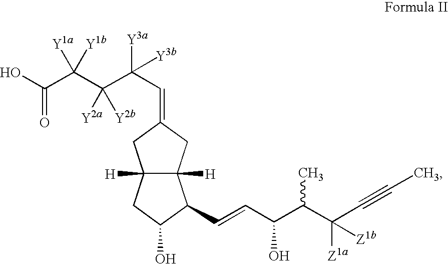

or a pharmaceutically acceptable salt thereof, wherein:
each Y is independently selected from hydrogen or deuterium;
each Z is independently selected from hydrogen and deuterium; and
at least one Y or Z is deuterium.

In one embodiment of Formula II, Y^{1a} and Y^{1b} are the same. In a more specific embodiment, Y^{1a} and Y^{1b} are simultaneously deuterium.

In another embodiment of Formula II, Y^{2a} and Y^{2b} are the same. In a more specific embodiment, Y^{2a} and Y^{2b} are simultaneously deuterium.

In yet another embodiment of Formula II, Z^{1a} and Z^{1b} are the same. Even more specifically, Z^{1a} and Z^{1b} are simultaneously deuterium.

In one particular embodiment, Y^{1a}, Y^{1b}, Y^{2a}, and Y^{2b} are simultaneously deuterium. In one aspect of this embodiment, Y^{1a}, Y^{1b}, Y^{2a}, and Y^{2b} are simultaneously deuterium

In another embodiment, the compound is selected from any one of the compounds set forth in the following table:

TABLE 2
Exemplary Compounds of Formula II

| Cmpd | Y^{1a} | Y^{1b} | Y^{2a} | Y^{2b} | Y^{3a} | Y^{3b} | Z^{1a} | Z^{1b} |
|---|---|---|---|---|---|---|---|---|
| 110 | D | D | H | H | D | D | H | H |
| 111 | H | H | D | D | D | D | H | H |
| 112 | D | D | D | D | D | D | H | H |
| 113 | D | D | H | H | D | D | D | D |
| 114 | H | H | D | D | D | D | D | D |
| 115 | D | D | D | D | D | D | D | D |

In another set of embodiments, any atom not designated as deuterium in any of the embodiments of Formula I or II set forth above is present at its natural isotopic abundance.

In still another embodiment, the compound is selected from:

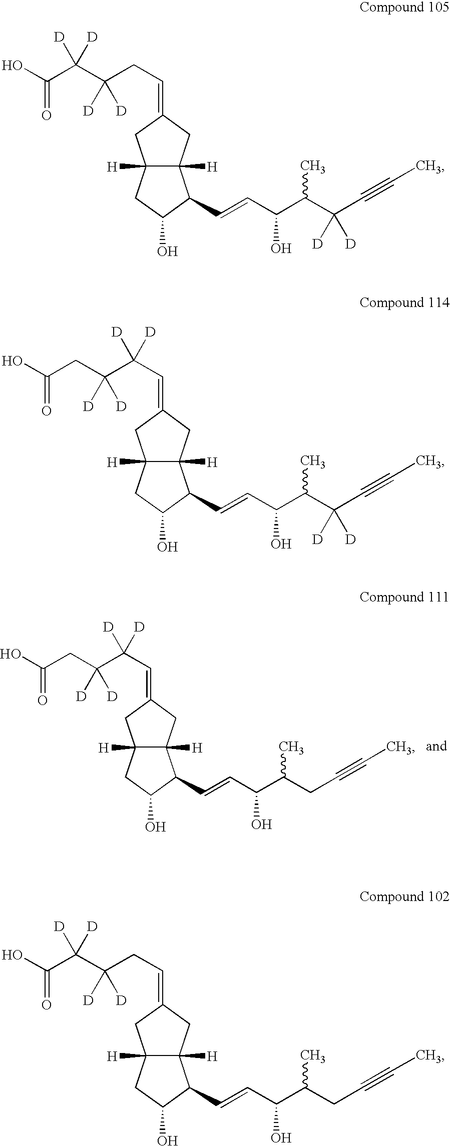

or a pharmaceutically acceptable salt of any of the foregoing compounds.

Preferred compounds are those where Y^{1a}, Y^{1b}, Y^{2a}, and Y^{2b} are simultaneously deuterium, such as Compounds 102 and 105. Compounds 102 and 105 differ from iloprost by having a high level of deuterium incorporation at the positions designated as D and a natural abundance of isotopes at all other atoms. Compounds where Y^{1a}, Y^{1b} Y^{2a}, and Y^{2b} are simultaneously deuterium may be obtained as described below where the level of deuterium incorporation is at least 90% at each position designated as D. Such chemical structure modifications are particularly beneficial in improving the metabolic stability of the present compounds relative to iloprost. Accordingly, one embodiment of the invention relates to a deuterated iloprost where Y^{1a}, Y^{1b}, Y^{2a}, and Y^{2b} are simultaneously deuterium, preferably with at least 90% deuterium incorporation at each position. In such compounds, all other atoms may be at natural abundance or one or more hydrogen atoms may be optionally replaced by deuterium.

The metabolic stability that deuteration of the upper side chain confers on iloprost-based compounds, as described herein, can be applied to other iloprost compounds. For example, W. Skuballa et. al., J. Med. Chem. 1986, 29(3), 313-315 reports certain iloprost analogues that have nearly identical profile of action and potency. These authors modify iloprost in the bottom side chain by replacing the 13-14 double bond by a triple bond, introduce a further methyl at C-20 and prepare the (S)-diastereomer at C-16. Accordingly, one embodiment of this invention relates to a compound of formula III:

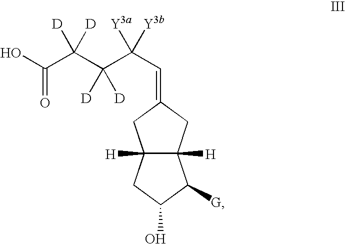

or a pharmaceutically acceptable salt thereof, wherein:
Y^{3a} and Y^{3b} are selected from hydrogen or deuterium;

### G is

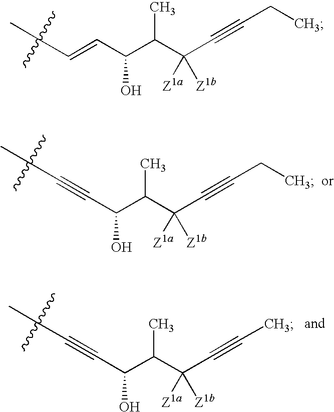

Z^{1a} and Z^{1b} are selected from hydrogen or deuterium. One embodiment provides compounds of formula III where Y^{3a} and Y^{3b} are the same and Z^{1a} and Z^{1b} are the same. When Z^{1a} and Z^{1b} are the same, there are two chiral centers in the G group. The chiral center bearing the CH₃ may be predominantly in the (S) configuration, or a 50/50 mixture of (R) and (S) stereochemistry.

The synthesis of compounds of Formulae I, II and III can be readily carried out by synthetic chemists of ordinary skill. Relevant procedures and intermediates are disclosed, for instance, in Gais H J et al., Chemistry 2006, 12(21): 5610-5617; Kramp G J et al, J Am Chem Soc 2005, 127(50): 17910-17920; Kim, M et al, J Org Chem 2006, 71(12):4642-4650; van Bergen, M et al, J Am Chem Soc 2002, 124(16): 4321-4328; Ueno K et al, Chem Pharm Bull 1984, 32(9): 3768-3769; Gais, H J et al, Tet Lett 1988, 29(15): 1773-1774; and U.S. Pat. Nos. 4,400,393 and 5,200,530.

Such methods can be carried out utilizing deuterated and optionally, other isotope-containing reagents and/or intermediates to synthesize the compounds delineated herein, or invoking standard synthetic protocols known in the art for introducing isotopic atoms to a chemical structure. The schemes below illustrate how the present compounds may be prepared.

A convenient method for producing a compounds of the Formula I is exemplified according to Scheme 1:

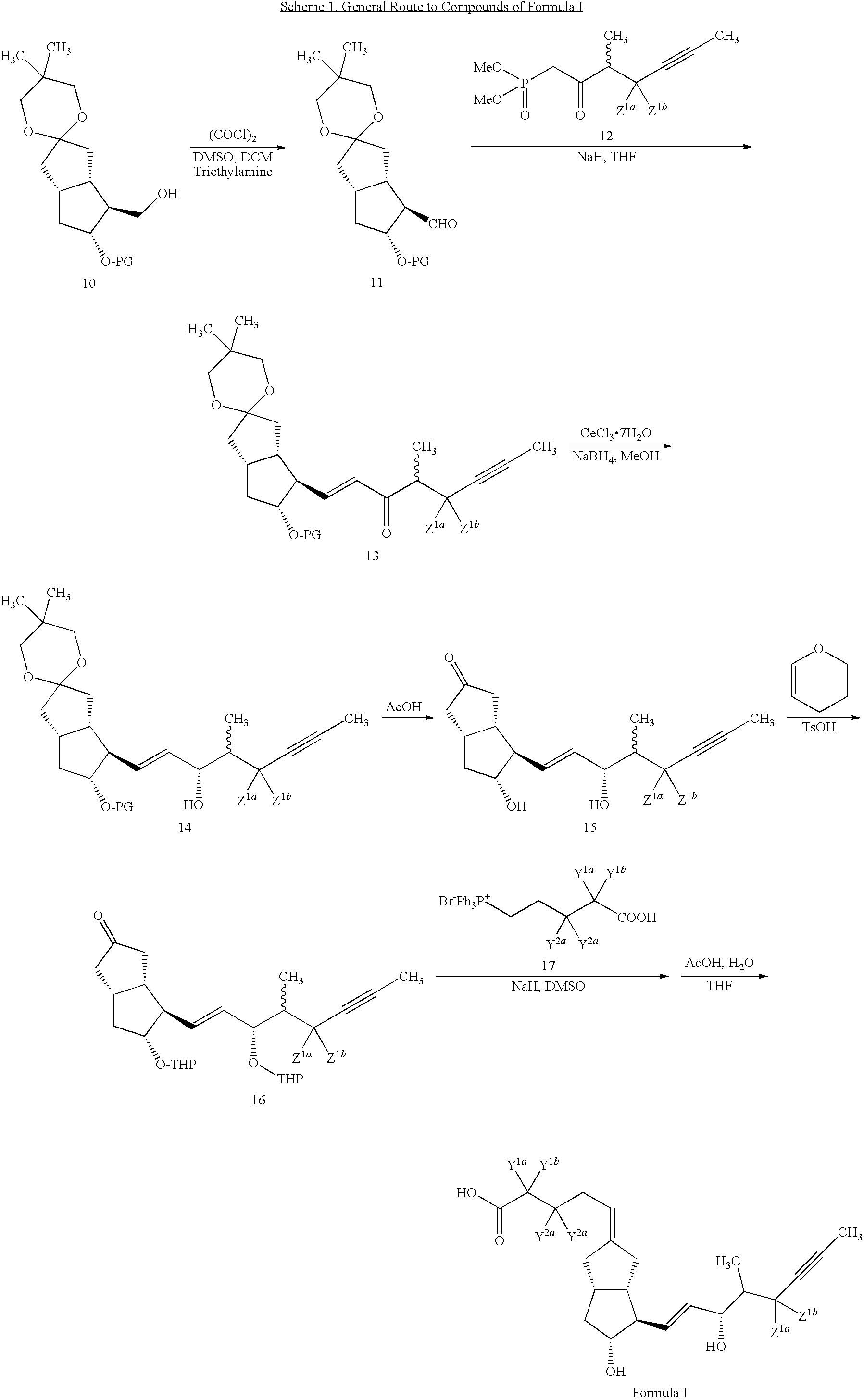

The synthesis of iloprost is described in U.S. Pat. No. 5,200,530 and the references cited therein. “PG” represents a protecting group, such as a silyl ether protecting group, examples of which include t-butyldimethylsilyl, dimethylphenylsilyl or dimethylthexylsilyl (see, for example, T. W. Greene and P. G. M. Wuts, Protective Groups in Organic Synthesis, 3^{rd} Ed., John Wiley and Sons (1999)). Scheme 1 shows how the general route to iloprost may be adapted to provide compounds of the present invention. The primary alcohol 10 is oxidized to the aldehyde by first reacting with oxalyl chloride and DMSO in DCM (dichloromethane, methylene chloride, CH₂Cl₂) at −60° C. followed by addition of triethylamine and warming to 0° C. The desired aldehyde 11 is then reacted with the dimethylphosphonate 12 in THF using sodium hydride as the base to provide 13. The ketone is reduced to the alcohol 14 in methanol at −40° C. using sodium borohydride and cerium chloride heptahydrate. Excess borohydride reagent is quenched with acetone to yield 14. The ketal and silyl ether protecting groups are removed by treatment with acid and tetrabutylammonium fluoride, respectively, to yield 15. The secondary alcohols are protected as the THP ethers by reaction with dihydropyran with toluene sulfonic acid as a catalyst to yield 16. The ketone is then reacted with the ylide of the appropriately substituted triphenyl pentanoic acid in DMSO with sodium hydride as base. Subsequent removal of the THP protecting groups with mild acid yields compounds of Formula I.

The appropriately deuterated iloprost can be prepared in a manner analogous to that described in Japanese Patent Application 2001309366. A starting material for 12 is prepared as shown below in Scheme 2a and described by Schulte, K E et al., Chem. Ber. 1954 87 p. 964-970. The preparation of intermediate 12 is described in the following Scheme 2b.

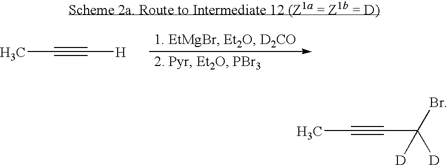

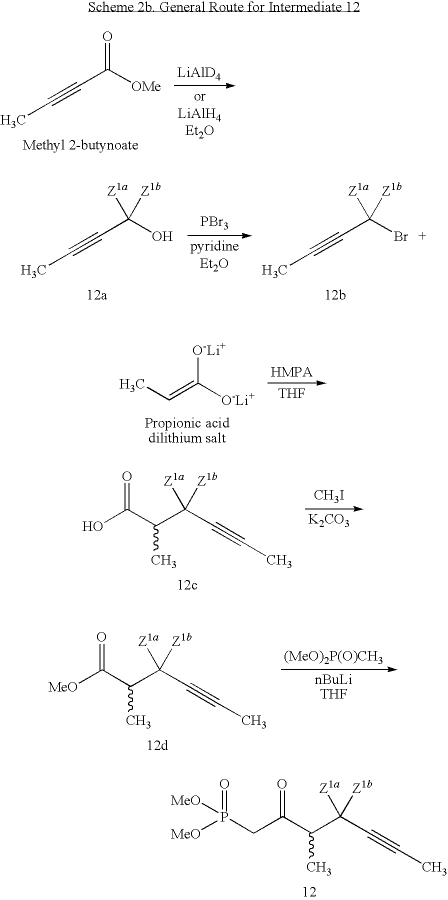

Compounds of Formula II can be synthesized according to Scheme 3.

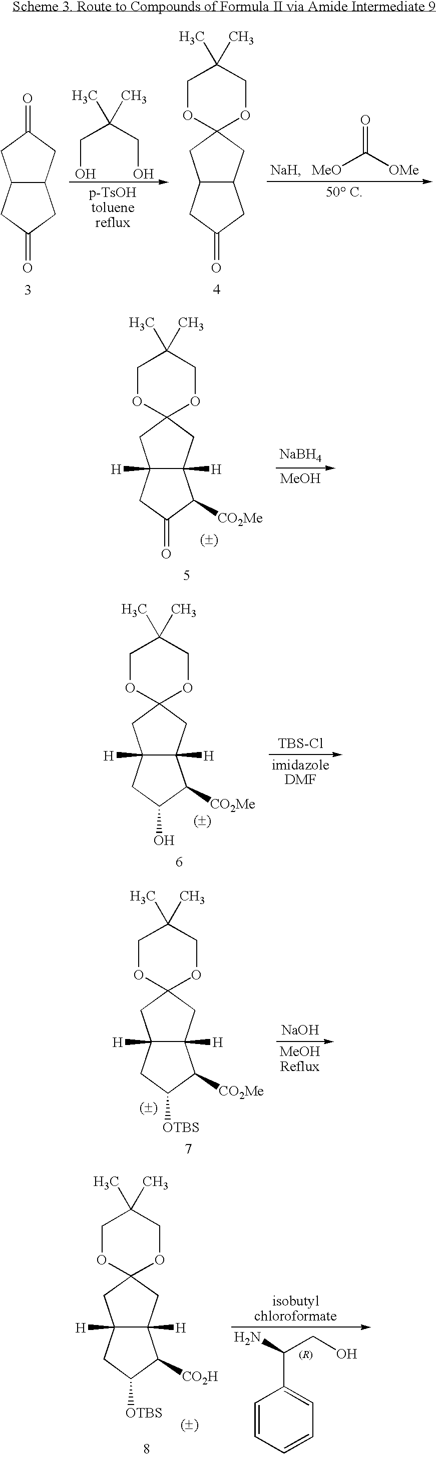

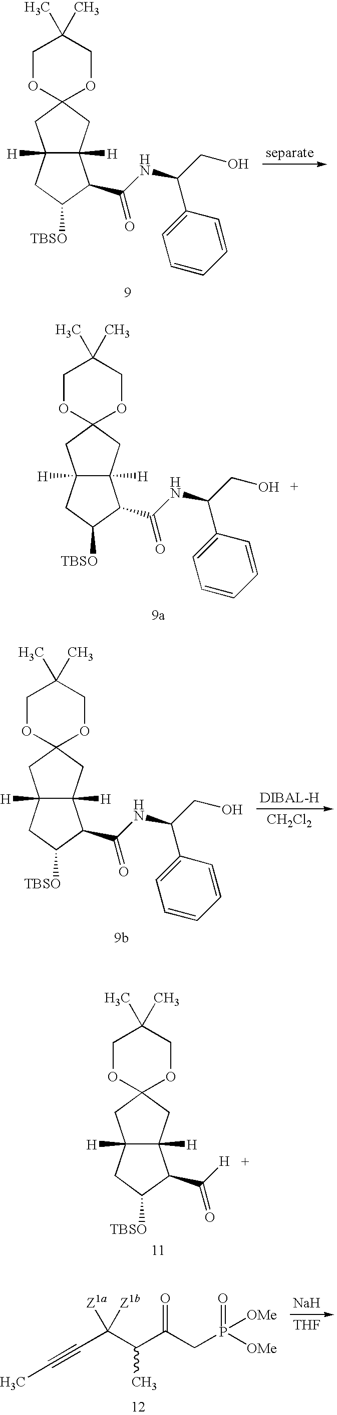

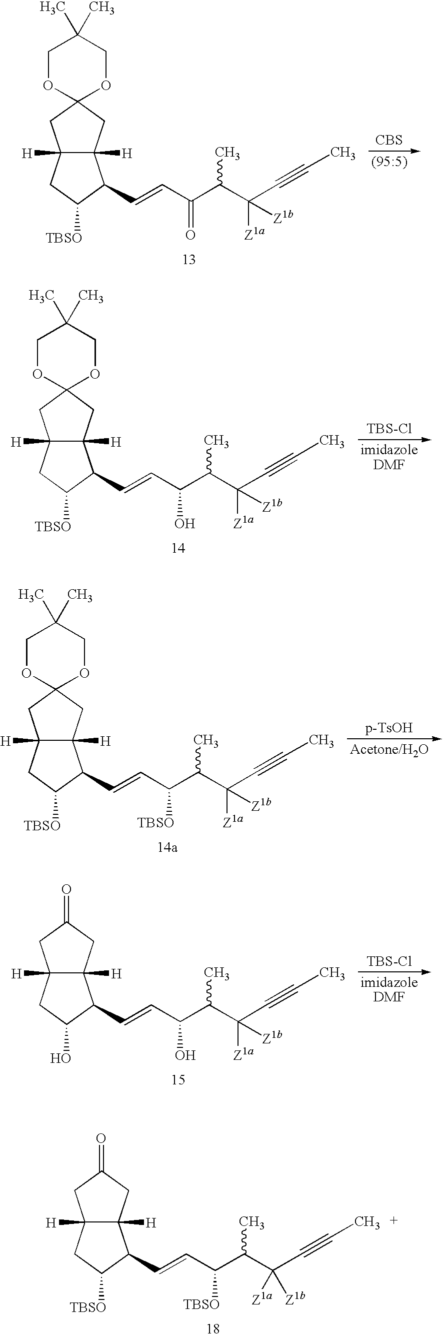

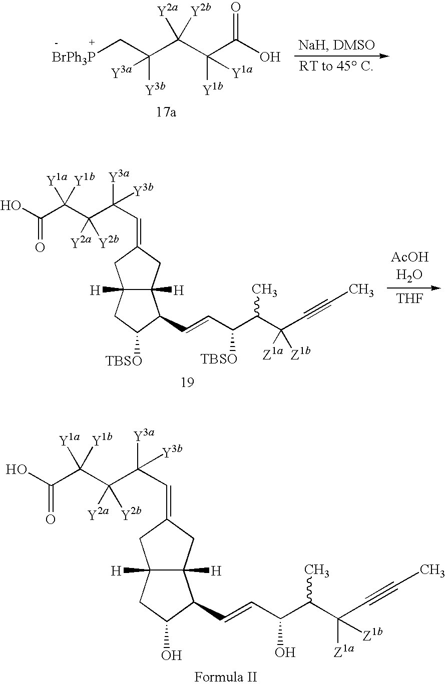

Scheme 3 above is based on chemistry that is known for the corresponding non-deuterated analogs and, in particular, on the process described in U.S. Pat. No. 4,925,956. which reports the preparation of optically active aldehyde 11 from the amide 9. According to the patent, the diastereomers corresponding to 9a and 9b are separated by column chromatography. The desired diastereomer 9b is then reduced with diisobutylaluminum hydride (Dibal-H) to provide optically active 11. The Wittig reagent 17a wherein Y^{1a}═Y^{1b}═Y^{2a}═Y^{2b}=D (17b) can be prepared as shown in Example 6 below, and the Wittig reagent 17a wherein Y^{2a}═Y^{2b}═Y^{3a}═Y^{3b}=D (17c) can be prepared as shown in Example 9 below. Compounds may be prepared wherein the level of deuterium incorporation at each Y¹ and Y² position is greater than 90%. In a typical preparation, the level of deuterium incorporation at each Y¹ and Y² position is at least 95%, with each Y² position having at least 98% deuterium.

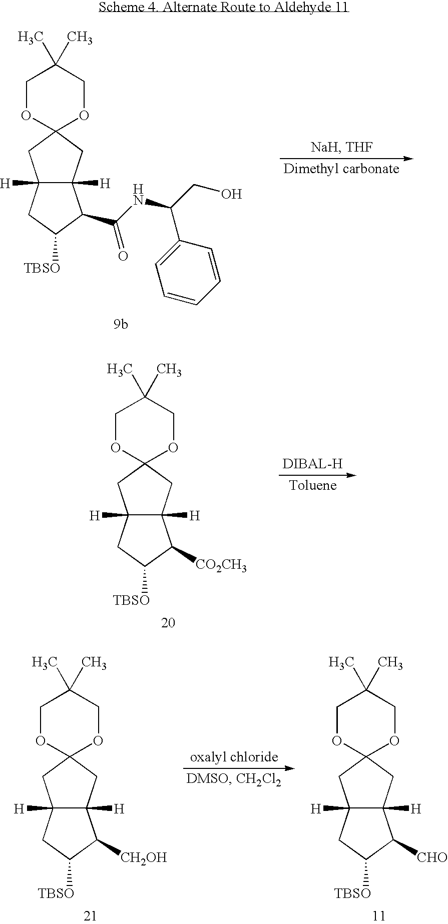

Scheme 4 above shows another approach to aldehyde 11. The amide 9b may be converted to the methyl ester 20, which then may be reduced with DIBAL-H to the alcohol 21. Treatment of 21 with oxalyl chloride and DMSO furnishes the aldehyde 11.

Other approaches to synthesizing compounds of the formulae herein (e.g., Formula I or II) can readily be adapted from references cited herein. Variations of these procedures and their optimization are within the skill of the ordinary practitioner.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., Y^{1a}, Y^{1b}, Y^{2a}, Y^{2b}, Z^{1a}, or Z^{1b}) or not. The suitability of a chemical group in a compound structure for use in synthesis of another compound structure is within the knowledge of one of ordinary skill in the art.

Additional methods of synthesizing compounds of formulae I, II and III and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Combinations of substituents and variables envisioned by this invention are only those that result in the formation of stable compounds.

The invention further provides a mixture of a compound of this invention and its lighter isotopologues. These mixtures may occur, for instance, simply as the result of an inefficiency of incorporating the isotope at a given position; intentional or inadvertent exchange of protons for deuterium, e.g. exchange of bulk solvent for heteroatom-attached deuterium; or intentional mixtures of pure compounds.

In one embodiment, such mixtures comprise at least about 50% of the heavy atom isotopic compound (i.e., less than about 50% of lighter isotopologues). More preferable is a mixture comprising at least 80% of the heavy atom isotopic compound. Most preferable is a mixture comprising 90% of the heavy atom isotopic compound. In one aspect, is a mixture at least about “X” % of the heavy atom isotopic compound (i.e., less than about X % of lighter isotopologues), where X is a number between 0 and 100, inclusive.

The synthetic schemes and examples herein describe certain deuterated compounds that are useful as synthetic intermediates for making compounds of Formula I or Formula II. Thus the invention also provides such a compound that is selected from the following:

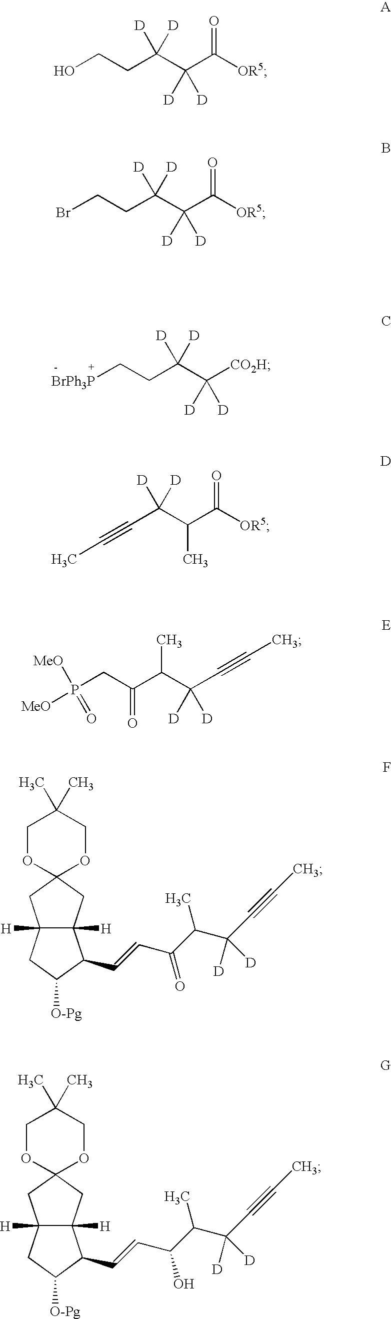

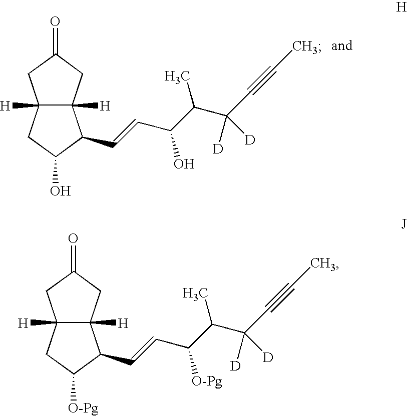

where R⁵ is hydrogen, deuterium, or a C₁-C₈ group and Pg is an alcohol protecting group. Examples of the C₁-C₈ group include, but are not limited to, C₁-C₆ alkyl such as methyl, ethyl, propyl and aralkyl such as benzyl. Examples of the Pg group include, but are not limited to, tert-butyldimethylsilyl (TBS), triisopropylsilyl (TIPS) and tetrahydropyranyl (THP).

### Compositions

The invention also provides compositions comprising an effective amount of a compound of Formula I, II and III, or a pharmaceutically acceptable salt thereof; and an acceptable carrier. In one embodiment, the composition is pyrogen-free. In another embodiment the composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets and sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain preferred embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

Preferred dosage forms include inhalable microparticle formulations, such as those formulations of iloprost which are used with the I-neb™ AAD® System or the Prodose® AAD® System, as well as those described for iloprost in PCT patent publication WO2006014930; and oral formulations, such as those described in United States patent publication US20050101673.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of the present invention further comprises a second therapeutic agent. The second therapeutic agent includes any compound or therapeutic agent known to have or that demonstrates advantageous properties when administered with vascular dilators of systemic or pulmonary arterial vascular beds or by platelet aggregation inhibitors. Such agents include those indicated as being useful in combination with iloprost which are described in detail in PCT patent publications WO1988001867; WO2005030187: WO2006014930: WO2005009446: WO2004019952; WO2000002450; WO1992013537; WO1997006806: and WO1998037894: and in United States Patent publications US20020128314; US20030139372; US20030162824; US20030216474; US20040033223; US20040052760; US 20040058940; US20040266880; US20050009847; US20050070596; US 20050080140; US20050101673; US20050106151; US20050119330; US20050239719; US20050239842; US20050239867; US20060183684; US20060160213.

In one embodiment, the second therapeutic agent is an agent useful in the treatment or prevention of a disease or condition selected from pulmonary arterial hypertension, Raynaud's phenomenon secondary to systemic sclerosis, contrast-mediated nephropathy, or lung cancer.

Even more specifically, the second therapeutic agent co-formulated with a compound of this invention is an agent useful in the treatment of pulmonary arterial hypertension.

In one embodiment, the second therapeutic agent is selected from a phosphodiesterase V inhibitor or an endothlin-1 antagonist. In another embodiment, the phosphodiesterase V inhibitor is sildenafil. In still another embodiment, the endothlin-1 antagonist is bosentan.

In another embodiment, the invention provides separate dosage forms of a compound of this invention and a second therapeutic agent that are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardsley, N.Y., 1970, 537. An effective unit dose amount of a compound of this invention can range from about 1 mg/kg body weight to about 500 mg/kg weight, more preferably 1 mg/kg to about 250 mg/kg, more preferably 1 mg/kg to about 75 mg/kg. Unit doses can be administered from once to nine times per day. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

It is expected that some of the second therapeutic agents referenced above will act synergistically with the compounds of this invention. When this occurs, its will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

According to another embodiment, the invention provides a method of treating a subject suffering from or susceptible to a disease that is beneficially treated by iloprost comprising the step of administering to said subject an effective amount of a compound or a composition of this invention. Such conditions and diseases are well known in the art and include embolism-linked and other skin diseases, pulmonary hypertension, fibrosis-related diseases such as scleroderma, cerebral malaria, poor venous flow, bone diseases (such as bone marrow edema, osteonecrosis and osteoarthritis) syncytial virus infection, pruritic or atopic symptoms, inflammatory disorders, CNS disorders, as well as others disclosed in US 20050080140; US20030139372; US20030216474; US20040266880; US20050009847; US20050101673; WO1988001867; WO1992013537; WO2000002450; WO2004019952; and WO2006014930.

In a preferred embodiment, the method of this invention is used to treat a subject suffering from or susceptible to a disease or condition selected from pulmonary arterial hypertension, Raynaud's phenomenon secondary to systemic sclerosis, contrast-mediated nephropathy, or lung cancer. Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In another embodiment, the invention provides a method of modulating the activity of a prostacyclin receptor in a cell comprising contacting the cell with one or more compounds of any of the formulae herein.

In another embodiment, the above method of treatment comprises the further step of co-administering to the patient one or more second therapeutic agents. The choice of second therapeutic agent may be made from any second therapeutic agent known to be useful for co-administration with iloprost. Such agents are specifically include any of those set forth above for use in pharmaceutical combinations of the invention.

In particular, the combination therapies of this invention include: treatment of erectile dysfunction in combination with a 15-hydroxyprostaglndindehydrogenase inhibitor; as an antithrombotic in combination with a betaine; treatment of angina, high blood pressure, pulmonary hypertension, congestive heart failure, chronic obstructive pulmonary disease (COPD), pulmonary heart disease, right ventricular failure, atherosclerosis, permeability conditions of reduced cardiovascular patency, peripheral vascular illnesses, cerebral apoplexy, bronchitis, allergic asthma, chronic asthma, allergic rhinitis, glaucoma, irritable bowel syndrome, tumors, kidney failure, cirrhosis of the liver and for treating male or female sexual problems each in combination with a phosphodiesterase V inhibitor; treating an inflammation related cardiovascular condition in combination with a COX-1 or COX-2 inhibitor; increasing or maintaining hair thickness in combination with a 15-hydroxyprostaglndindehydrogenase inhibitor; treating multiple sclerosis in combination with a cannabidiol derivative; treating a bacterial infection in combination with an α1-antitrypsin or serine protease inhibitor; treating a lung proliferative vascular disorder in combination with a HMG-CoA reductase inhibitor; treating pulmonary hypertension in combination with thalidomide or a phosphodiesterase IV inhibitor; treating hypertension, complications in diabetes and metabolic syndrome in combination with a blood pressure lowering agent; or treating pulmonary arterial hypertension in combination with an endothelin receptor antagonist, a phosphodiesterase inhibitor or a calcium channel blocker.

Other combination therapies useful in this invention are those combination therapies that employ iloprost and which are, described in US 20020128314; US 20040033223; US 20040058940; US20030162824; US20040052760; US20050070596; US20050106151; US20050119330; US20050239719; US20050239842; US20050239867; US20060160213; US20060183684; WO1997006806: WO1998037894: WO2005009446: and WO2005030187.

In a specific embodiment, the invention provides a method of treating a patient suffering from pulmonary arterial hypertension and comprises the step of co-administering to the patient a compound of Formula I, II or III and a second therapeutic agent selected from a phosphodiesterase V inhibitor or an endothelin-1 antagonist. In a more specific embodiment, the phosphodiesterase V inhibitor is sildenafil. In another more specific embodiment, the endothelin-1 antagonist is bosentan.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention comprising both a compound of the invention and a second therapeutic agent to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of formula I alone or together with one or more of the above-described second therapeutic agents in the manufacture of a medicament, either as a single composition or as separate dosage forms, for treatment or prevention in a subject of a disease, disorder or symptom set forth above. Another aspect of the invention is a compound of the formulae herein for use in the treatment or prevention in a subject of a disease, disorder or symptom thereof delineated herein.

### Diagnostic Methods and Kits

The compounds and compositions of this invention are also useful as reagents in methods for determining the concentration of iloprost in solution or biological sample such as plasma, examining the metabolism of iloprost and other analytical studies.

According to one embodiment, the invention provides a method of determining the concentration, in a solution or a biological sample, of iloprost, comprising the steps of:
a) adding a known concentration of a compound of Formula I or II to the solution of biological sample;
b) subjecting the solution or biological sample to a measuring device that distinguishes iloprost from a compound of Formula I or II;
c) calibrating the measuring device to correlate the detected quantity of the compound of Formula I or II with the known concentration of the compound of Formula I or II added to the biological sample or solution; and
d) measuring the quantity of iloprost in the biological sample with said calibrated measuring device; and
e) determining the concentration of iloprost in the solution of sample using the correlation between detected quantity and concentration obtained for a compound of Formula I or II.

Measuring devices that can distinguish iloprost from the corresponding compound of Formula I or II include any measuring device that can distinguish between two compounds that differ from one another only in isotopic abundance. Exemplary measuring devices include a mass spectrometer, NMR spectrometer, or IR spectrometer.

In another embodiment, the invention provides a method of evaluating the metabolic stability of a compound of Formula I or II comprising the steps of contacting the compound of Formula I or II with a metabolizing enzyme source for a period of time and comparing the amount of the compound of Formula I or II with the metabolic products of the compound of Formula I or II after the period of time.

In a related embodiment, the invention provides a method of evaluating the metabolic stability of a compound of Formula I or II in a patient following administration of the compound of Formula I or II. This method comprises the steps of obtaining a serum, urine or feces sample from the patient at a period of time following the administration of the compound of Formula I or II to the subject; and comparing the amount of the compound of Formula I or II with the metabolic products of the compound of Formula I or II in the serum, urine or feces sample.

The present invention also provides kits for use to treat pulmonary arterial hypertension, Raynaud's phenomenon secondary to systemic sclerosis, contrast-mediated nephropathy, or lung cancer. These kits comprise: a) a pharmaceutical composition comprising a compound of Formula I, II or III or a salt thereof; or a prodrug, or a salt of a prodrug thereof; or a hydrate, solvate, or polymorph thereof, wherein said pharmaceutical composition is in a container; and b) instructions describing a method of using the pharmaceutical composition to treat said disease.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. Preferably, the container is a blister pack.

The kit may additionally comprise a memory aid of the type containing information and/or instructions for the physician, pharmacist or subject. Such memory aids include numbers printed on each chamber or division containing a dosage that corresponds with the days of the regimen which the tablets or capsules so specified should be ingested, or days of the week printed on each chamber or division, or a card which contains the same type of information. For single dose dispensers, memory aids further include a mechanical counter which indicates the number of daily doses that have been dispensed and a battery-powered micro-chip memory coupled with a liquid crystal readout and/or audible reminder signal which, for example, reads out the date that the last daily dose has been taken and/or reminds one when the next dose is to be taken. Other memory aids useful in such kits are a calendar printed on a card, as well as other variations that will be readily apparent.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### SYNTHETIC EXAMPLES

The synthetic examples shown below provided key intermediates for preparing the compounds of this invention.

### Example 1

### Synthesis of (3α′S,4′R,5′R,6α′R)-5′-(tert-butyldimethylsilyloxy)-5,5-dimethylhexahydro-1′H-spiro[[1,3]dioxane-2,2′-pentalene]-4′-carbaldehyde (11)

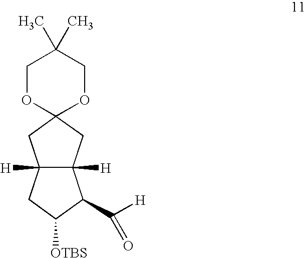

### Step 1. (2,2-dimethyltrimethylenedioxy)-cis-bicyclo[3.3.0]octan-3,7-dione (4)

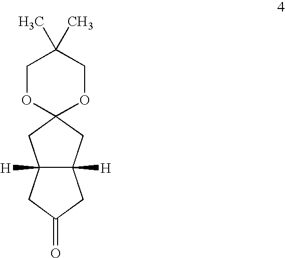

To the solution of the cis-Bicyclo [3.3.0] octan-3,7-dione, 3 (25 g, 181.06 mmol) in toluene (300 mL) was added 2,2-dimethyl-1, -propanediol (18.9 g, 181.06 mmol) and p-toluenesulfonic acid monohydrate (catalytic amount) and the solution was stirred at room temperature overnight. The reaction mixture was concentrated and the crude was subjected to column chromatography to give the monoprotected ketone, 4 (17.8 g, 44%). ¹HNMR (300 MHz, CDCl₃): δ 0.94 (s, 6H), 1.80 (dd, 2H), 2.13-2.70 (m, 6H), 2.80-2.90 (m, 2H), 3.52 (s, 2H), 3.65 (s, 2H). MS (M+H): 225.

### Step 2. (±) (2,2-Dimethyltrimethylenedioxy)-cis-bicyclo[3.3.0]octan-3-one-2-carboxylic acid methyl ester (5)

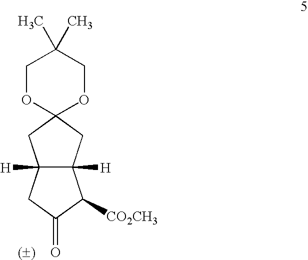

To a suspension of sodium hydride (2.33 g, 53.5 mmol) in dimethyl carbonate (80 mL) was added the mono-protected ketone, 4 (10 g, 44.6 mmol) dissolved in dimethyl carbonate (20 mL) and the solution was stirred at 50° C. overnight. The mixture was cooled, the excess sodium hydride was quenched with methanol and the mixture was neutralized with acetic acid. The product was extracted with dichloromethane (3×50 mL) and concentrated under vacuum to give the crude product. The crude was purified by column chromatography to give the product, 5. (7.0 g, 56%). ¹H NMR (300 MHz, CDCl₃): δ 0.93 (s, 6H), 1.50-1.90 (m, 3H), 2.04-2.10 (m, 1H), 2.20-2.60 (m, 4H), 3.30 (d, 1H), 3.44-3.59 (m, 4H), 3.77 (s, 3H), 10.35 (b s, 1H). MS (M+H): 283.

### Step 3. (±) 7,7-(2,2-Dimethyltrimethylenedioxy)-3-α-hydroxy-cis-bicyclo[3.3.0]octane-2-β-carboxylic acid methyl ester (6).

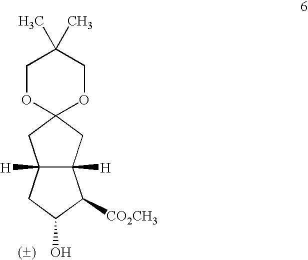

To the solution of the methyl ester, 5 (7 g, 24.8 mmol) in methanol (80 mL) at −40° C. was added NaBH₄ (1.87 g, 49.6 mmol) and the solution was stirred for 2 hours at the same temperature. Acetone (2 mL) was added to the reaction mixture and the solution was neutralized with satd. oxalic acid (5 mL). The solvents were evaporated and the residue was extracted with dichloromethane (2×25 mL). The organic layer was dried over sodium sulfate and evaporated to give the product, 6. (5 g, 71%). ¹H NMR (300 MHz, CDCl₃): δ 0.96 (s, 6H), 1.52-1.72 (m, 1H), 1.90-2.04 (m, 1H), 2.09-2.30 (m, 4H), 2.43-2.90 (m, 3H), 3.40-3.56 (m, 4H), 3.72 (s, 3H), 4.20-4.30 (m, 1H). MS (M+H): 285.

### Step 4. (±) 7,7-(2,2-Dimethyltrimethylenedioxy)-3-α-tert-butyldimethylsilyloxyy-cis-bicyclo[3.3.0]octane-2-β-carboxylic acid methyl ester (7).

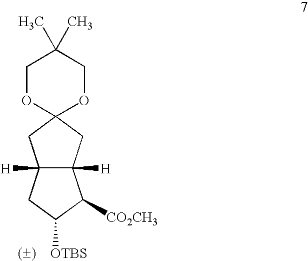

To a solution of the hydroxyl compound, 6 (3 g, 10.6 mmol) in DMF (40 mL) was added imidazole (1.72 g, 25.32 mmol) and tert-butyldimethylchlorosilane (1.91 g, 12.66 mmol) and the mixture was stirred at room temperature overnight. Water (10 mL) was added to the reaction mixture and the solution was extracted with ether (2×25 mL). The organic layer was dried over Na₂SO₄, evaporated and purified by column chromatography to give the product, 7. (3 g, 71%). ¹H NMR (300 MHz, CDCl₃) δ: 0.02 (s, 3H), 0.05 (s, 3H), 0.85 (s, 9H), 0.93 (s, 6H), 1.60-1.74 (m, 1H), 1.90-2.04 (m, 2H), 2.09-2.18 (m, 3H), 2.44-2.50 (m, 1H), 2.56-2.62 (m, 2H), 3.40-3.60 (m, 4H), 3.76 (s, 3H), 4.20-4.30 (m, 1H). MS (M+H): 399.

### Step 5. (±) 7,7-(2,2-Dimethyltrimethylenedioxy)-3-α-tert-butyldimethylsilyloxyy-cis-bicyclo[3.3.0]octane-2-β-carboxylic acid (8).

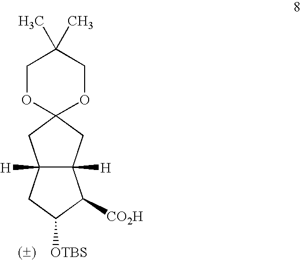

To the solution of the ester, 7 (3 g, 7.52 mmol) was added methanol (40 mL) and 5% NaOH (8.2 mL) and the solution was refluxed for 1.5 h. The solution was concentrated under vacuum, diluted with water (20 mL) and extracted with diethyl ether (25 mL). The mixture was cooled in an ice bath, acidified (pH=3) with 2N H₂SO₄ and extracted with diethyl ether (2×25 mL). The ether layer was dried over Na₂SO₄ and evaporated to give the acid, 8. (2.0 g, 69%). ¹HNMR (300 MHz, CDCl₃): δ 0.02 (s, 3H), 0.05 (s, 3H), 0.81 (s, 9H), 0.95 (s, 6H), 1.60-1.76 (m, 1H), 1.90-2.03 (m, 2H), 2.09-2.18 (m, 3H), 2.44-2.50 (m, 1H), 2.56-2.62 (m, 2H), 3.40-3.60 (m, 4H), 4.20-4.30 (m, 1H). MS (M+H): 385.

### Step 6. 7,7-(2,2-Dimethyltrimethylenedioxy)-3-α-tert-butyldimethylsilyloxyy-cis-bicyclo[3.3.0]octane-2-β-carboxylic acid D-(-)-α-phenylglycinolamide (9b)

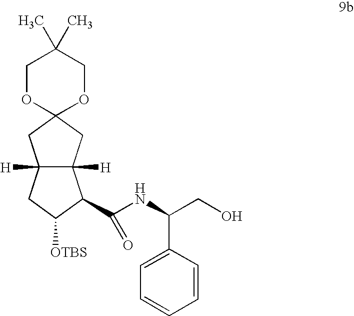

To the solution of the acid, 8 (4 g, 10.4 mmol) in acetone (30 mL), NEt₃ (1.3 mL, 9.32 mmol) was added and the solution was stirred for 5 minutes at 0° C. Isobutylchloroformate (1.2 mL, 8.73 mmol) dissolved in acetone (15 mL) was added to the reaction mixture and the solution was stirred for 20 minutes at the same temperature. D-(-)-α-phenylglycinol (1.17 g, 8.56 mmol) dissolved in acetone (15 mL) and acetonitrile (15 mL) was added dropwise to the reaction mixture and the solution is stirred for 24 hours at room temperature. The reaction mixture was concentrated, the residue was dissolved in dichloromethane (25 mL) and washed with brine (2×10 mL). The organic layer was dried over Na₂SO₄ and evaporated. The diastereomers were separated using column chromatography (Hexane:EtOAc=3:1) to give the required diastereomer, 9b. (1.82 g, 34%). ¹H NMR (300 MHz, CDCl₃): δ 0.06 (s, 6H), 0.83 (s, 9H), 0.95 (s, 6H), 1.42-1.90 (m, 1H), 1.90-2.03 (m, 2H), 2.09-2.18 (m, 3H), 2.44-2.50 (m, 1H), 2.56-2.62 (m, 2H), 3.40-3.60 (m, 4H), 3.88-3.96 (m, 2H), 4.14-4.23 (m, 1H), 5.05-5.17 (m, 1H), 6.50 (d, 1H), 7.26-7.34 (m, 5H). MS (M+H) m/z: 504. [α]_{D}=−27.39 (0.54 in CHCl₃).

### Step 7. Methyl (3a′S, 4′R, 5′R, 6a′R)-5′-(tert-butyldimethylsilyloxy)-5,5-dimethyl hexahydro-1′H spiro[[1,3]dioxane-2,2′pentalene]-4′carboxylate (20)

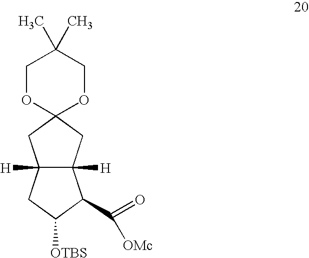

To a suspension of sodium hydride (60% in mineral oil, 2.33 g, 66.9 mmol) in tetrahydrofuran (60 mL) was added the amide 9b (10.0 g, 44.6 mmol)) dissolved in dimethyl carbonate (50 mL) at 0° C. and the solution was stirred at room temperature for 2 hours. The mixture was cooled, the excess sodium hydride was quenched with methanol and the mixture was neutralized with acetic acid. The product was extracted with methylene chloride (3×50 mL) and concentrated under vacuum to give the crude product. The crude was purified by column chromatography to give the product. (7.00 g, 56%). ¹H NMR (400 MHz, CDCl₃) δ: 0.02 (s, 3H), 0.08 (s, 3H), 0.85 (s, 9H), 0.99 (s, 3H), 1.0 (s, 3H), 1.50-1.56 (m, 1H), 1.80-1.95 (m, 2H), 2.09-2.28 (m, 3H), 2.45-2.50 (m, 1H), 2.60-2.70 (m, 2H), 3.40 (d, J=4.4 Hz, 2H), 3.50 (d, J=4.4 Hz, 2H), 3.68 (s, 3H), 4.24-4.30 (m, 1H). MS (M+H): 399.

### Step 8. (3a′S, 4′R, 5′R, 6a′R)-5′-(tert-butyldimethylsilyloxy)-5,5-dimethyl hexahydro-1′H spiro[[1,3]]dioxane-2,2′pentalene]-4′-yl]methanol (21)

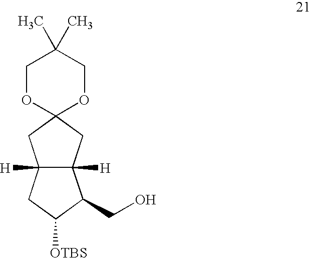

DIBAL-H 1.0 M in toluene (15.0 mmol, 15.0 mL) was added to a solution of methyl ester 20 from the previous step (3.00 g, 7.53 mmol) in methylene chloride (3 mL) at 78° C. and the solution was stirred for 2 hours at the same temperature. Methanol (2 mL) was added to the reaction mixture which was followed by the addition of aqueous potassium tartrate (1 mL). Then the mixture was filtered through Celite and was washed with methylene chloride (25 mL). The combined organic phases were dried over Na₂SO₄ and concentrated under vacuum to give the crude product. The crude was purified by column chromatography to give the product. (1.70 g, 70%). ¹H NMR (400 MHz, CDCl₃) δ: 0.05 (s, 3H), 0.07 (s, 3H), 0.87 (s, 9H), 0.93 (s, 3H), 0.98 (s, 3H), 1.40-1.58 (m, 1H), 1.73-2.01 (m, 3H), 2.01-2.20 (m, 3H), 2.20-2.42 (m, 1H), 3.42-3.49 (m, 4H), 3.64-3.67 (m, 2H), 3.82-3.90 (m, 1H). MS (M+H): 371.

### Step 9. (3a′S, 4′R, 5′R, 6a′R)-5′-(tert-butyldimethylsilyloxy)-5,5-dimethyl hexahydro-1′H spiro[[1,3]]dioxane-2,2′pentalene]-4′carbaldehyde (11)

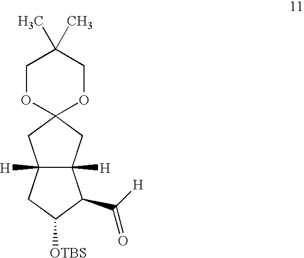

Oxalyl chloride (0.17 mL, 2.02 mmol) was dissolved in 5 mL of dichloromethane, cooled to 60° C. and mixed with dimethyl sulfoxide (0.21 mL, 4.05 mmol) in 2 mL of dichloromethane. After 10 minutes a solution of 0.50 g (1.35 mmol) of alcohol 21 from the previous step in dichloromethane (5 mL) was added and the reaction mixture was stirred at room temperature for 45 minutes. Triethylamine (0.58 mL, 4.18 mmol) in methylene chloride (2 mL) was added to the reaction mixture and the solution was warmed to room temperature. Water (20 mL) was added, and the organic phase was separated and washed with brine (2×10 mL). The combined organic phases were dried over Na₂SO₄ and concentrated under vacuum to give the crude product. (0.48 g, 96%). ¹H NMR (400 MHz, CDCl₃): δ 0.03 (s, 3H), 0.06 (s, 3H), 0.90 (s, 9H), 0.95 (s, 3H), 0.98 (s, 3H), 1.60-1.70 (m, 1H), 1.80-1.90 (m, 2H), 2.10-2.30 (m, 3H), 2.40-2.60 (m, 1H), 2.70-2.90 (m, 2H), 3.46-3.50 (m, 4H), 4.24-4.30 (m, 1H), 9.69 (d, J=2.4 Hz, 1H). MS (M+H) m/z: 369.

### Example 2

### Synthesis of 3-methyl-2-oxohept-5-ynylphosphonic acid dimethyl ester 12 (Z^{1a}=Z^{1b}=H)

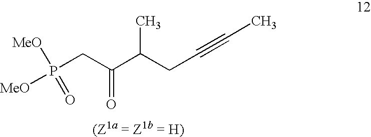

### Step 1. 2-methylhex-4-ynoic acid 12c (Z^{1a}=Z^{1b}=H)

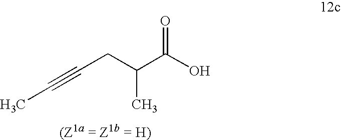

To a solution of diisopropylamine (32.75 mL, 233.09 mmol) in THF (100 mL) at −50° C. was added 1.6 M n-BuLi in hexane (94 mL) and the solution was stirred for 5 minutes. The reaction mixture was allowed to warm to −20° C. and the mixture was treated with a mixture of HMPA (15.7 mL) and propionic acid (6.75 mL, 90.23 mmol) dropwise. The reaction mixture was stirred at room temperature for 30 minutes. The contents were then cooled to 0° C. and 1-bromo-2-butyne, 12b (Z^{1a}=Z^{1b}=H) (10 g, 75.19 mmol) in THF (20 mL) was added to the reaction mixture and stirred at room temperature for 2 hours. The contents were poured into 10% HCl (20 mL) and the solution was extracted with ether (3×25 mL). The organic layer was dried over Na₂SO₄ and evaporated to give the product, 12c (Z^{1a}=Z^{1b}=H). (12 g). The crude was directly taken to next step. ¹H NMR (300 MHz, CDCl₃): δ 1.15 (d, 3H), 1.77 (t, 3H), 2.35 (m, 2H), 2.66 (m, 1H)

### Step 2. Methyl 2-methylhex-4-ynoate 12d (Z^{1a}=Z^{1b}=H)

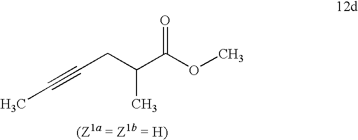

To the solution of the crude acid, 12c (Z^{1a}=Z^{1b}=H) (12 g, 95.1 mmol) in acetone (100 mL) was added MeI (8.9 mL, 142.68 mmol) and K₂CO₃ (26.3 g, 190.24 mmol) and the solution was stirred at room temperature overnight. The reaction mixture was evaporated and the contents were dissolved in water (25 mL). The solution was extracted with ether (3×25 mL), and the organic layer was dried over Na₂SO₄ and evaporated. The crude product was vacuum distilled to give pure product, 12d (Z^{1a}=Z^{1b}=H). (4.4 g, 33%). ¹H NMR (300 MHz, CDCl₃): δ 1.25 (d, 3H), 1.77 (t, 3H), 2.34 (m, 2H), 2.66 (m, 1H), 3.69 (s, 3H).

### Step 3. 3-methyl-2-oxohept-5-ynylphosphonic acid dimethyl ester, 12 (Z^{1a}=Z^{1b}=H)

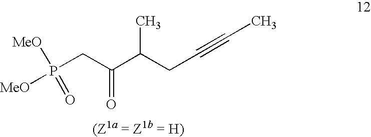

To a solution of dimethyl methylphosphonate (4.5 mL, 42.80 mmol) in THF (20 mL) was added 1.6 M n-BuLi in hexane (24 mL) dropwise and the solution was stirred at −78° C. for 30 minutes. The ester, 12d (Z^{1a}=Z^{1b}=H) (3.0 g, 21.40 mmol) dissolved in THF (10 mL) was added to the reaction mixture dropwise and the mixture was stirred at −78° C. for 3 hours and at ambient temperature for 1 h. The reaction mixture was quenched with acetic acid (1 mL) added with saturated brine (30 mL), and was extracted with ether (3×10 mL). The ether layer was dried over Na₂SO₄ and evaporated to give the crude product. The crude product was vacuum distilled to give the pure product, 12 (Z^{1a}=Z^{1b}=H). (2 g, 40%). ¹H NMR (300 MHz, CDCl₃): δ 1.18 (d, 3H), 1.76 (t, 3H), 2.36 (m, 2H), 2.64 (m, 1H), 3.26 (d, 2H), 3.77 (s, 3H), 3.81 (s, 3H)

### Example 3

### Synthesis of 4,4-d2-3-methyl-2-oxohept-5-ynylphosphonic acid dimethyl ester 12 (Z^{1a}=Z^{1b}=D)

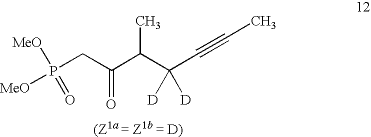

### Step 1. 1,1-d₂-But-2-yn-1-ol 12a (Z^{1a}=Z^{1b}=D)

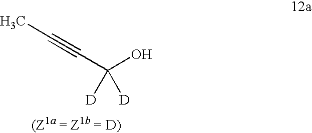

To a suspension of lithium aluminum deuteride (1.28 g, 30.57 mmol) in ether (60 mL) was added dropwise methyl 2-butynoate (5 g, 51 mmol) in ether (20 mL) at 0° C. The reaction mixture was stirred was stirred for 1 hour at room temperature and quenched with satd. ammonium chloride (1 mL). The ether layer was filtered, dried over Na₂SO₄ and evaporated. The residue was vacuum distilled to give the alcohol, 12a (Z^{1a}=Z^{1b}=D). (2 g, 55%). ¹H NMR (300 MHz, CDCl₃): 1.85 (s, 3H)

### Step 2. 1,1-d₂-1-Bromo-but-2-yne 12b (Z^{1a}=Z^{1b}=D)

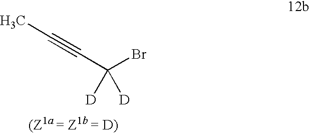

To a stirred solution of 1,1-d₂-but-2-yn-1-ol, 12a (Z^{1a}=Z^{1b}=D) (1.2 g, 16.64 mmol) in ether (10 mL) at 0° C. was added pyridine (4 mL, 49.92 mmol), and phosphorous tribromide (0.89 mL, 11.15 mmol) dropwise and the solution was warmed to reflux for 2 hours. The reaction mixture was cooled to 0° C., the contents were treated with satd. NaBr solution (10 mL) and extracted with ether (2×10 mL). The ether layer was dried over Na₂SO₄ and evaporated to give the product, 12b (Z^{1a}=Z^{1b}=D). (0.6 g, 30%). ¹H NMR (400 MHz): 1.88 (s, 3H)

### Step 3. 3,3-d₂-2-Methylhex-4-ynoic acid 12c (Z^{1a}=Z^{1b}=D)

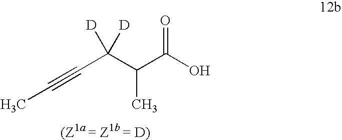

To a solution of diisopropylamine (1.93 mL, 13.77 mmol) in THF (10 mL) at −50° C. was added 1.2M n-BuLi in hexane (7.4 mL) and the solution was stirred for 5 minutes. The reaction mixture was allowed to warm to −20° C. and the mixture was treated with a mixture of HMPA (0.77 mL) and propionic acid (0.39 mL, 5.32 mmol) dropwise. The reaction mixture was stirred at room temperature for 30 minutes. The contents were then cooled to 0° C. and 1,1-d2-1-bromo-but-2-yne, 12b (Z^{1a}=Z^{1b}=D) (0.60 g, 4.44 mmol) was added to the reaction mixture and stirred at room temperature for 2 hours. The contents were poured into 10% HCl (5 mL) and the solution was extracted with ether (2×10 mL). The organic layer was dried over Na₂SO₄ and evaporated to give the product, 12c (Z^{1a}=Z^{1b}=D) (1.0 g). The crude was directly taken to next step. ¹H NMR (300 MHz, CDCl₃): δ 1.15 (d, 3H), 1.83 (s, 3H), 2.64 (q, 1H)

### Step 4. Methyl 3,3-d₂-Methylhex-4-ynoate 12d (Z^{1a}=Z^{1b}=D)

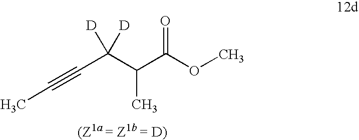

To the solution of the crude acid, 12c (Z^{1a} Z^{1b} D) (1.0 g, 11.53 mmol) in acetone (15 mL) was added MeI (1.07 mL, 17.30 mmol) and K₂CO₃ (3.18 g, 23.06 mmol) and the solution was stirred at room temperature overnight. The reaction mixture was evaporated and the contents were dissolved in water (10 mL). The solution was extracted with ether (2×10 mL) and the organic layer was dried over Na₂SO₄ and evaporated. The crude was purified by vacuum distillation to give the crude product, 12d (Z^{1a}=Z^{1b}=D) (0.6 g). The crude was directly taken to next step. ¹H NMR (300 MHz, CDCl₃): δ 1.15 (d, 3H), 1.84 (s, 3H), 2.65 (q, 1H), 3.69 (s, 3H)

### Step 5. 4,4-d₂-3-Methyl-2-oxohept-5-ynylphosphonic acid dimethyl ester 12 (Z^{1a}=Z^{1b}=D)

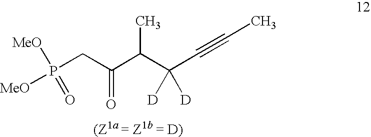

To a solution of dimethyl methylphosphonate (0.66 mL, 8.56 mmol) in THF (10 mL) was added 1.2 M n-BuLi in hexane (6.42 mL) dropwise and the solution was stirred at −78° C. for 30 minutes. The ester, 12d (Z^{1a}=Z^{1b}=D) (0.60 g, 4.28 mmol) dissolved in THF (5 mL) was added to the reaction mixture dropwise and the mixture was stirred at −78° C. for 3 hours and at ambient temperature overnight. The reaction mixture was quenched with acetic acid (0.5 mL) added with saturated brine (10 mL), and was extracted with ether (2×5 mL). The ether layer was dried over Na₂SO₄ and evaporated to give to give the crude product, 12 (Z^{1a}=Z^{1b}=D) (0.40 g).

### Example 4

### Synthesis of (3aS,4R,5R,6aR)-5-(tert-butyldimethylsilyloxy)-4-((3S,E)-3-(tert-butyldimethylsilyloxy)-4-methyloct-1-en-6-ynyl)hexahydropentalen-2(1H)-one (18)

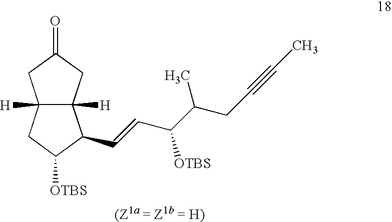

### Step 1. [(3a′S, 4′R, 5′R, 6a′R)-5′-(tert-Butyldimethylsilyloxy)-5,5-dimethyl hexahydro-1′H spiro[[1,3]]dioxane-2,2′pentalene]-4′-yl]-4-methyloct-1-en-6-yn-3-one (13) (Z^{1a}=Z^{1b}=H)

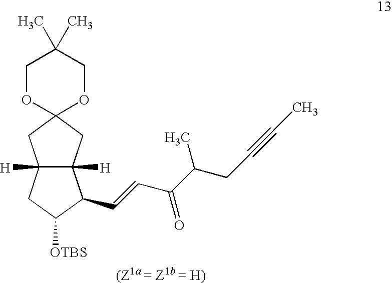

To a suspension of 55% sodium hydride (0.108 g, 2.71 mmol) in tetrahydrofuran (12 mL) was added dimethyl 3-methyl-2-oxohept-5-ynylphosphonate (Compound 12 where Z^{1a}=Z^{1b}=H) (0.630 g, 2.71 mmol) in tetrahydrofuran (8 mL). The solution was stirred for 30 minutes at room temperature and then a solution of aldehyde 11 from Example 1 (1.00 g, 2.71 mmol) in tetrahydrofuran (8 mL) was added. After 2 hours the reaction mixture was neutralized with acetic acid (0.20 mL) and concentrated under vacuum. The residue was taken up in methylene chloride (20 mL) and washed with brine solution (2×20 mL). The organic layer was dried over Na₂SO₄ and concentrated under vacuum to give the crude product. The crude was purified by column chromatography to give the titled product (0.90 g, 70%). ¹H NMR (400 MHz, CDCl₃): δ −0.03 (s, 3H), 0.01 (s, 3H), 0.85 (s, 9H), 0.95 (s, 3H), 0.98 (s, 3H), 1.27 (d, J=7.2 Hz, 3H), 1.40-1.66 (m, 1H), 1.70 (s, 3H), 1.70-1.90 (m, 2H), 2.10-2.50 (m, 8H), 2.80-2.90 (m, 1H), 3.46 (s, 2H), 3.49 (s, 2H), 3.80-3.90 (m, 1H), 6.20 (dd, J=3.2 Hz, 0.8 Hz, 1H), 6.70 (dd, J=15.6 Hz, 8.2 Hz, 1H).

### Step 2. (E)-[(3a′S, 4′R, 5′R, 6a′R)-5′-(tert-butyldimethylsilyloxy)-5,5-dimethyl hexahydro-1′H spiro[[1,3]]dioxane-2,2′pentalene]-4′-yl]-4-methyloct-1-en-6-yn-3-ol (14) (Z^{1a}=Z^{1b}=H)

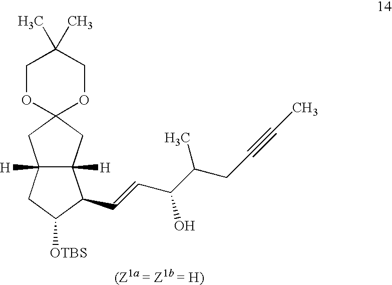

Compound 13 (Z^{1a}=Z^{1b}=H) (2.00 g, 4.22 mmol) was dissolved in methanol (58 mL) and cooled to −78° C. Cerium (III) chloride heptahydrate (1.58 g, 4.22 mmol) was added and the reaction mixture was stirred at approximately 78° C. for 1 h. Sodium borohydride (0.291 g, 7.59 mmol) was added to the reaction mixture at the same temperature and stirred for another 45 minutes at approximately 78° C. After addition of acetone (2 mL), the reaction mixture was slowly warmed to room temperature, neutralized with acetic acid (0.2 mL) and the solvent was evaporated under vacuum. The residue was dissolved in dichloromethane and washed with water. The organic layer was dried with Na₂SO₄ and concentrated under vacuum to give the crude product. The crude was purified by column chromatography to give the titled product. (2.00 g, 95%). ¹H NMR (400 MHz, CDCl₃): δ −0.03 (s, 3H), −0.01 (s, 3H), 0.80 (s, 9H), 0.90 (s, 3H), 1.00 (s, 3H), 1.30 (d, J=7.2 Hz, 3H), 1.30-1.60 (m, 4H), 1.60-1.80 (m, 2H), 1.80 (s, 3H), 1.90-2.30 (m, 6H), 2.30-2.50 (m, 1H), 3.45 (s, 2H), 3.50 (s, 2H), 3.70-3.80 (m, 1H), 3.90-4.00 (m, 0.45H), 4.10-4.20 (m, 0.55H), 5.40-5.60 (m, 2H)

### Step 3. (3aS,4R,5R,6aR)-5-Hydroxy-4-((3S,E)-3-hydroxy-4-methyloct-1-en-6-ynyl)hexahydropentalen-2(1H)-one (15) (Z^{1a}=Z^{1b}=H)

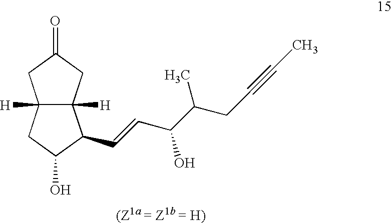

To a solution of acetal 14 (Z^{1a}=Z^{1b}=H) (0.50 g, 1.05 mmol) in acetone (4 mL) and H₂O (1.5 mL) was added p-toluenesulfonic acid (10 mg). The mixture was stirred at ambient temperature for 12 hours. Then aqueous NaHCO₃ (5 mL) was added and the aqueous phase was extracted with diethylether (2×10 mL). The combined organic phases were dried Na₂SO₄ and concentrated in vacuum to give crude product (0.500 g, 100%). ¹H NMR (400 MHz, CDCl₃): δ 1.30 (d, J=7.0 Hz, 3H), 1.40-1.80 (m, 2H), 1.80 (s, 3H), 2.08-2.60 (m, 7H), 2.60-2.70 (m, 1H), 2.70-2.90 (m, 1H), 3.90-4.20 (m, 2H), 5.40-5.60 (m, 2H).

### Step 4. (3aS,4R,5R,6aR)-5-(tert-Butyldimethylsilyloxy)-4-((3S,E)-3-(tert-butyldimethylsilyloxy)-4-methyloct-1-en-6-ynyl)hexahydropentalen-2(1H)-one (18) (Z^{1a}=Z^{1b}=H)

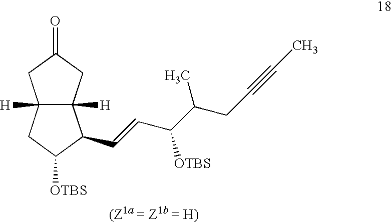

To a solution of 15 (Z^{1a}=Z^{1b}=H) (0.50 g, 1.80 mmol) in DMF (3 mL), imidazole (0.705 g, 10.8 mmol) was added. The mixture was stirred for 5 min at ambient temperature. Then tert-butyldimethylsilylchloride (0.660 g, 4.32 mmol) was added. The mixture was stirred at ambient temperature for 14 hours. Then aqueous NaHCO₃ (10 mL) was added and the aqueous phase was extracted with Et₂O (3×10 mL). The combined organic phases were dried Na₂SO₄ and concentrated in vacuum to give the crude product. The crude was purified by column chromatography to give the product (0.310 g, 55%). ¹H NMR (400 MHz, CDCl₃): δ −0.03 (s, 3H), −0.01 (s, 3H), 0.02 (s, 3H), 0.04 (s, 3H), 0.85 (s, 9H), 0.90 (s, 9H), 1.30 (s, 3H), 1.50-1.60 (m, 1H), 1.60-1.75 (m, 1H), 1.80 (s, 3H), 1.90-2.60 (m, 9H), 2.70-2.90 (m, 1H), 3.90-4.10 (m, 1.5H), 4.10-4.20 (m, 0.5H), 5.50-5.60 (m, 2H).

### Example 5

### Synthesis of (3aS,4R,5R,6aR)-5-(tert-Butyldimethylsilyloxy)-4-((3S,E)-3-(tert-butyldimethylsilyloxy)-5,5-d₂-4-methyloct-1-en-6-ynyl)hexahydropentalen-2(1H)-one (18) (Z^{1a}=Z^{1b}=D)

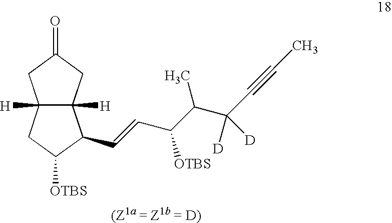

### Step 1. [(3a′S, 4′R, 5′R, 6a′R)-5′-(tert-Butyldimethylsilyloxy)-5,5-dimethyl hexahydro-1′H spiro[[1,3]]dioxane-2,2′pentalene]-4′-yl]-5,5-d₂—4-methyloct-1-en-6-yn-3-one (13) (Z^{1a}=Z^{1b}=D)

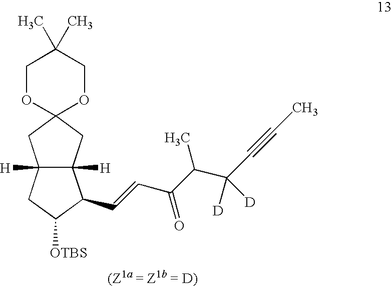

To a suspension of 55% sodium hydride (0.05 g, 1.30 mmol) in tetrahydrofuran (6 mL) was added 4,4-d2-3-methyl-2-oxohept-5-ynylphosphonic acid dimethyl ester (0.30 g, 1.30 mmol) in tetrahydrofuran (8 mL). The solution was stirred for 30 minutes at room temperature and then (3a′S, 4′R, 5′R, 6a′R)-5′-(tert-butyldimethylsilyloxy)-5,5-dimethyl hexahydro-1′H spiro[[1,3]]dioxane-2,2′pentalene]-4′carbaldehyde (11) (0.48 g, 1.30 mmol) dissolved in tetrahydrofuran (4 mL) was added. After 2 hours the reaction mixture was neutralized with acetic acid (0.10 mL) and concentrated under vacuum. The residue was taken up in methylene chloride (10 mL) and washed with brine (2×10 mL). The organic layer was dried over Na₂SO₄ and concentrated under vacuum to give the crude product. The crude was purified by column chromatography to give the product (0.80 g, 64%). ¹H NMR (400 MHz, CDCl₃): δ −0.01 (s, 3H), −0.03 (s, 3H), 0.85 (s, 9H), 0.95 (s, 3H), 0.99 (s, 3H), 1.27 (s, 3H), 1.44-1.54 (m, 1H), 1.77 (d, J=1.60 Hz, 3H), 1.79-1.83 (m, 2H), 2.10-2.22 (m, 3H), 2.25-2.34 (m, 1H), 2.38-2.52 (m, 2H), 2.87-2.91 (m, 1H), 3.47 (s, 2H), 3.49 (m, 1H), 3.81-3.89 (m, 1H), 6.20 (ddd, J=15.6 Hz, 2.8 Hz, 0.4 Hz, 1H), 6.70 (dd, J=15.6 Hz, 8.40 Hz, 1H).

### Step 2. (E)-[(3a′S, 4′R, 5′R, 6a′R)-5′-(tert-Butyldimethylsilyloxy)-5,5-dimethyl hexahydro-1′H spiro[[1,3]]dioxane-2,2′pentalene]-4′-yl]-5,5-d₂-4-methyloct-1-en-6-yn-3-ol (14) (Z^{1a}=Z^{1b}=D)

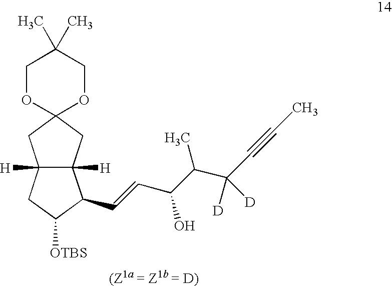

Compound 13 (Z^{1a}=Z^{1b}=D) (0.800 g, 1.68 mmol) was dissolved in methanol (23 mL) and cooled to approximately 78° C. Cerium (III) chloride heptahydrate (0.63 g, 1.68 mmol) was added and the reaction mixture was stirred at 78° C. for 1 hour. Sodium borohydride (0.116 g, 3.02 mmol) was added to the reaction mixture at the same temperature and stirred for another 45 minutes at 78° C. After addition of acetone (1 mL), the reaction mixture was slowly warmed to room temperature, neutralized with acetic acid (0.1 mL) and the solvent was evaporated under vacuum. The residue was dissolved in methylene chloride and washed with water. The organic layer was dried with Na₂SO₄ and concentrated under vacuum to give the crude product. The crude was purified by column chromatography to give the product. (0.60 g, 72%). ¹H NMR (400 MHz, CDCl₃): δ −0.03 (s, 3H), −0.01 (s, 3H), 0.90 (s, 9H), 0.95 (s, 3H), 1.00 (s, 3H), 1.50 (m, 1H), 1.60-1.80 (m, 3H), 1.80 (s, 3H), 2.10-2.30 (m, 6H), 2.30-2.40 (m, 1H), 3.50 (s, 4H), 3.70-3.80 (m, 1H), 3.90-4.00 (m, 1H), 4.00-4.10 (m, 1H), 5.40-5.70 (m, 2H).

### Step 3. (3aS,4R,5R,6aR)-4-((3R,E)-5,5-d₂-3-Hydroxy-4-methyloct-1-en-6-ynyl)-5-hydroxyhexahydropentalen-2(1H)-one (15) (Z^{1a}=Z^{1b}=D)

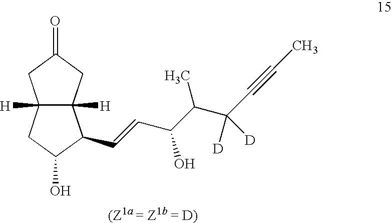

To a solution of Compound 14 (Z^{1a}=Z^{1b}=D) (1.80 g, 3.76 mmol) in acetone (14 mL) and H₂O (6 mL) was added p-toluenesulfonic acid (14 mg). The mixture was stirred at ambient temperature for 12 hours. Then aqueous NaHCO₃ (10 mL) was added and the aqueous phase was extracted with diethyl ether (2×10 mL). The combined organic phases were dried over Na₂SO₄ and concentrated in vacuum to give crude product (1.00 g, 100%). ¹H NMR (400 MHz, CDCl₃): δ 1.30 (d, J=7.0 Hz, 3H, 1.40-1.80 (m, 2H), 1.80 (s, 3H), 2.08-2.60 (m, 7H), 2.60-2.70 (m, 1H), 2.70-2.90 (m, 1H), 3.90-4.00 (m, 1.30H), 4.10-4.20 (m, 0.7H), 5.40-5.60 (m, 2H).

### Step 4. (3aS,4R,5R,6aR)-5-(tert-Butyldimethylsilyloxy)-4-((3S,E)-3-(tert-butyldimethylsilyloxy)-5,5-d₂-4-methyloct-1-en-6-ynyl)hexahydropentalen-2(1H)-one (18) (Z^{1a}=Z^{1b}=D)

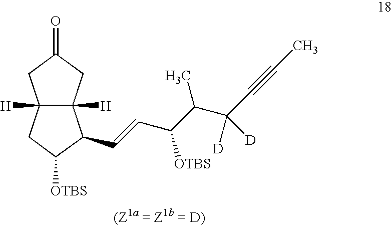

To a solution of Compound 15 (Z^{1a}=Z^{1b}=D) (1.00 g, 3.59 mmol) in DMF (6 mL), imidazole (1.41 g, 21.5 mmol) was added. The mixture was stirred for 5 min at ambient temperature. Then tert-butyldimethylsilylchloride (1.32 g, 8.97 mmol) was added. The mixture was stirred at ambient temperature for 14 hours. Then aqueous NaHCO₃ (10 mL) was added and the aqueous phase was extracted with diethyl ether (3×10 mL). The combined organic phases were dried over Na₂SO₄ and concentrated in vacuum to give the crude product. The crude was purified by column chromatography to give the product. (1.30 g, 72%). ¹H NMR (400 MHz, CDCl₃): δ −0.03 (s, 3H), −0.01 (s, 3H), 0.02 (s, 3H), 0.04 (s, 3H), 0.85 (s, 9H), 0.90 (s, 9H), 1.30 (s, 3H), 1.50-1.60 (m, 1H), 1.70-1.75 (m, 1H), 1.80 (s, 3H), 1.90-2.60 (m, 9H), 2.70-2.90 (m, 1H), 3.90-4.20 (m, 2H), 5.50-5.60 (m, 2H).

### Example 6

### Synthesis of (4-Carboxy-3,3,4,4-d₄-butyl)triphenylphosphonium bromide (17b) (Y^{1a/1b}═Y^{2a/2b}=D; Y^{3a/3b}═H)

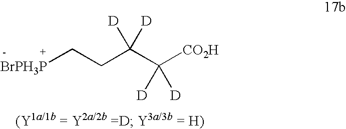

### Step 1. Ethyl 2,2,3,3-d₄-5-Hydroxypentanoate

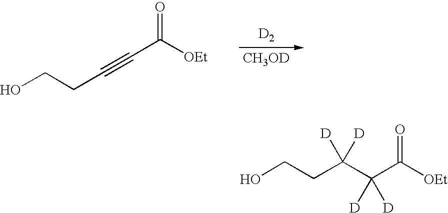

A solution of ethyl 5-hydroxypent-2-ynoate (16.0 g, 106.6 mmol, prepared according to the procedure described in J. Chem. Soc. Perkin Trans. I 1999, 2852-2863) in CH₃OD (80 mL) was subjected to deuterogenation conditions using deuterium gas (Isotec) at room temperature overnight. The reaction mixture was monitored by GC-MS. The reaction mixture was filtered through a Celite pad and the solution was evaporated to give the crude deuterated product (14.0 g, 82%) which was directly taken to next step. ¹H NMR (400 MHz, CDCl₃) δ: 1.25 (t, J=7.2 Hz, 3H), 3.60 (t, J=6.4 Hz, 2H), 3.80 (t, J=6.4 Hz, 2H), 4.20 (q, J=7.2 Hz, 2H).

### Step 2. 3,3,4,4-d₄-Tetrahydro-2H-pyran-2-one

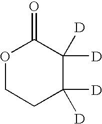

To a solution of ethyl 5-hydroxypentanoate-2,2,3,3-d4 (14.0 g, 93.3 mmol) in benzene (800 mL) was added anhydrous pTSA (p-toluenesulfonic acid) (10 mg) and the solution was heated to reflux using a Dean-Stark apparatus for 8 hours. The solution was cooled to room temperature and the reaction mixture was quenched with solid NaHCO₃. The reaction mixture was filtered and evaporated to give the crude product (10.0 g, 100%). ¹H NMR (400 MHz, CDCl₃): δ 1.80 (t, J=6.0 Hz, 2H), 4.20 (t, J=6.0 Hz, 2H)

### Step 3. 2,2,3,3-d₄-5-Bromopentanoic acid

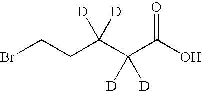

To a solution of BBr₃ (9.10 mL, 95.9 mmol) in methylene chloride (200 mL) was added δ-valerolactone-2,2,3,3-d4 (10.0 g, 95.9 mmol) in methylene chloride and the solution was stirred at room temperature overnight. The reaction mixture was quenched with D₂O (10 mL) and the solution was stirred at room temperature for 1 hour. The reaction mixture was mixed with water (50 mL) and was extracted with methylene chloride (2×25 mL), and the organic layer was dried over Na₂SO₄ and evaporated to give the titled product (3.50 g, 20%). ¹H NMR (400 MHz, CDCl₃): δ 1.90 (t, J=6.4 Hz, 2H), 3.40 (t, J=6.4 Hz, 2H).

### Step 4. (4-Carboxy-3,3,4,4-d₄-butyl)triphenylphosphonium bromide (17b) (Y^{1a/1b}═Y^{2a/2b}=D; Y^{3a/3b}═H)

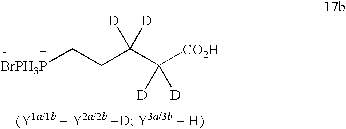

To a solution of 2,2,3,3-d₄-5-bromopentanoic acid (2.16 g, 11.72 mmol) in CD₃CN (23 mL), was added triphenylphosphine (3.07 g, 11.72 mmol, 1.0 equiv). The mixture was heated to reflux for a period of 15 hours then cooled to ambient temperature. The cooled solution was concentrated to one-half volume then diluted with Et₂O until a cloudy mixture was obtained. Crystallization was initiated by scratching the inside wall of the flask which resulted in the formation of a white precipitate. The mixture was filtered and the material washed with Et₂O. The pure material was lyophilized to remove trace solvents and afforded a white solid (2.76 g, 64%) of the titled product. MS (M+H): 367.1.

### Example 7

### Synthesis of (E)-2,2,3,3-d₄-5-((3aS,4R,5R,6aS)-5-Hydroxy-4-((S,E)-3-hydroxy-4-methyloct-1-en-6-ynyl)hexahydropentalen-2(1H)-ylidene)pentanoic acid (Compound 102)

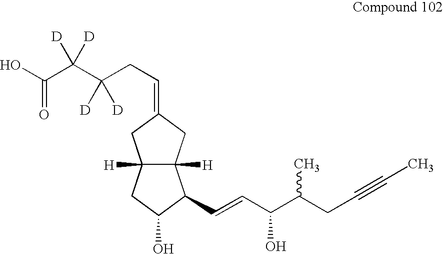

### Step 1. (E)-5-((3aS,4R,5R,6aS)-5-(tert-butyldimethylsilyloxy)-4-((S,E)-3-(tert-butyldimethylsilyloxy)-4-methyloct-1-en-6-ynyl)hexahydropentalen-2(1H)-ylidene)-2,2,3,3-tetradeuteropentanoic acid (19) (Y^{1a/1b}═Y^{2a/2b}=D; Y^{3a/3b}=Z^{1a/1b}═H)

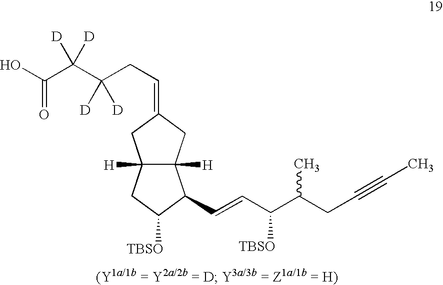

Two round-bottom flasks were flame-dried under nitrogen. One flask was charged with (4-carboxy-3,3,4,4-tetradeuterobutyl)triphenylphosphonium bromide 17b (815 mg, 1.82 mmol, see Example 6 for preparation) and then flushed with nitrogen. The compound was suspended in benzene to form a slurry, the flask was fitted onto a rotary evaporator, and the benzene was removed in vacuo. (Note that for this procedure, gas-tight plastic syringes were used to add all liquid reagents.) A nitrogen balloon was attached to the rotary evaporator and the flask was back-filled with nitrogen. The azeotrope procedure described below was repeated twice and then the flask was charged with a stir bar, placed in a vacuum desiccator containing Drierite and subjected to high vacuum for about 30 minutes. During this time, the second round-bottom flask was charged with 18 (Z^{1a}=Z^{1b}=H) (181 mg, 0.358 mmol, see Example 4 for preparation) and then flushed with nitrogen. Compound 18 (Z^{1a}=Z^{1b}=H) was subjected to the azeotrope procedure described below three times, and then the flask was charged with a stir bar, placed in a vacuum desiccator and subjected to high vacuum for about 45 minutes. During this time the flask containing 17b was placed under nitrogen and dry THF (10.1 mL from a new bottle, ≦99.9%, inhibitor-free, Sigma-Aldrich) was added. To the resulting suspension was added t-BuOK (3.94 mL, 3.94 mmol, 1M solution in THF). The resulting red, cloudy mixture was stirred rapidly for 30 min. The flask containing Compound 18 (Z^{1a}=Z^{1b}=D) was placed under nitrogen. THF (3.59 mL) was added, and the resulting solution was added quickly via canula to the solution containing 17b. This was followed with a THF rinse (1.76 mL). After stirring overnight, the reaction was quenched with 50% citric acid in D₂O and diluted with ethyl acetate. The organic layer was washed twice with 50% citric acid in D₂O. The combined aqueous solutions were washed twice with ethyl acetate. The combined organic solutions were dried (Na₂SO₄), filtered and concentrated. Purification on an ISCO instrument (40 g SiO₂, 12.5% EtOAc in heptanes) afforded 180 mg (85%) of titled product. ¹H NMR (300 MHz, C₆D₆) of mixture of diastereomers (only chemical shifts of major diastereomer are reported): δ 5.66 (m, 1H), 5.54 (m, 1H), 5.16 (br s, 1H), 4.20 (td, J=55.5, 5.2, 1H), 3.74 (q, J=8.1, 1H), 2.55-2.31 (m, 2H), 2.31-1.95 (m, 8H), 1.95-1.83 (m, 3H), 1.61 (s, 3H), 1.32 (m, 2H), 1.24-1.12 (m, 3H), 1.04 (s, 9H), 1.01 (s, 9H), 0.17 (s, 3H), 0.17 (s, 3H), 0.13 (s, 3H), 0.10 (s, 3H). MS (M−H): 590.9.

Azeotrope procedure used in Example 7, Step 1. The material to be azeotroped was dissolved in benzene and the flask fitted onto a roto-evaporator (“rotovap”) (about 5 mL benzene per 180 mg of material.) The bath temperature was about 20° C. The initial pressure was set at 80 torr, and then the pressure was lowered in about 10 torr increments to a minimum of about 7 torr. A balloon filled with nitrogen was attached to an inlet valve on the rotavap, so that when the evaporation was complete the flask could be back-filled with nitrogen rather than air.

### Step 2. (E)-2,2,3,3-d₄-5-((3aS,4R,5R,6aS)-5-Hydroxy-4-((S,E)-3-hydroxy-4-methyloct-1-en-6-ynyl)hexahydropentalen-2(1H)-ylidene)pentanoic acid (Compound 102)

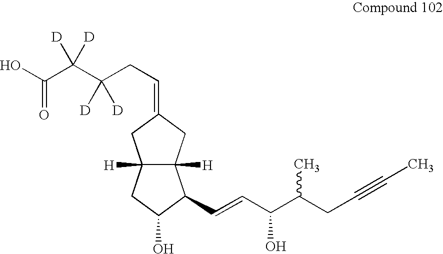

A 50-mL round-bottomed flask was charged with Compound 19 (Y^{1a}═Y^{1b}═Y^{2a}═Y^{2b}=D; Y^{3a}═Y^{3b}=Z^{1a}=Z^{1b}=H) (217 mg, 0.366 mmol) anhydrous THF (3.7 mL), and tetrabutylammonium fluoride (1.0 M solution in THF, 2.19 mL). The reaction mixture was stirred for 60 hours with monitoring by TLC analysis. The reaction mixture was diluted with ethyl acetate (5 mL), treated with D₂O (5 mL), and adjusted to pH=3 with 5% DCl in D₂O solution. The aqueous layer was extracted with ethyl acetate (3×25 mL) and the combined organic extracts were concentrated to an oil. The oil was purified by column chromatography on silica gel (10 cm×1.5 cm; eluent 5% MeOH in CH₂Cl₂) to deliver Compound 102 as a yellow oil and a portion of Compound 102 as a mixture of EIZ isomers (77 mg). The oil containing the desired isomer was dissolved in ethyl acetate (15 mL) and washed with 10% DCl in D₂O solution (3×3 mL) to deliver Compound 102 as a pale yellow oil (22 mg, 17%). The oil containing a mixture of EIZ isomers was dissolved in ethyl acetate (15 mL) and washed with 10% DCl in D₂O solution (3×3 mL). The organic layer was concentrated to an oil and purified by column chromatography on silica gel (10 cm×1.5 cm; eluent 3% MeOH in CH₂Cl₂) to deliver further Compound 102 as an oil (40 mg, 30% yield). HPLC (Column: Waters SunFire Prep Silica, 5 μm, 4.6×250 mm column; Mobile Phase: 96:4 hexanes/i-PrOH (1.5 mL/min); Wavelength: 254 nm): retention time: 12.32/13.06 minutes (Z) and 17.21/18.26 minutes (E); 89.6% purity (E). ¹H NMR (300 MHz, C₆D₆) of mixture of diastereomers (only chemical shifts of major diastereomer are reported): δ 5.70-5.61 (m, 1H), 5.54-5.41 (m, 1H), 5.16 (br s, 1H), 4.20 (dt, J₁=20.0, J₂=7.8, 1H), 3.67 (q, J=9.1, 1H), 2.46-1.90 (m, 12H), 1.58-1.56 (m, 3H), 1.32-0.88 (m, 7H). MS (M−H) 363.2.

### Example 8

### Synthesis of (E)-2,2,3,3-d₄-5-((3aS,4R,5R,6aS)-4-((R,E)-5,5-d₂-3-Hydroxy-4-methyloct-1-en-6-ynyl)-5-hydroxyhexahydropentalen-2(1H)-ylidene)pentanoic acid, (Compound 105)

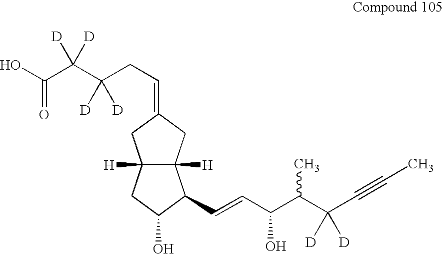

### Step 1. (E)-5-((3aS,4R,5R,6aS)-5-(tert-butyldimethylsilyloxy)-4-((S,E)-3-(tert-butyldimethylsilyloxy)-5,5-d₂-4-methyloct-1-en-6-ynyl)hexahydropentalen-2(1H)-ylidene)-2,2,3,3-d₄-pentanoic acid (19) (Y^{1a/1b}═Y^{2a/2b}=Z^{1a/1b}=D; Y^{3a/3b}═H)

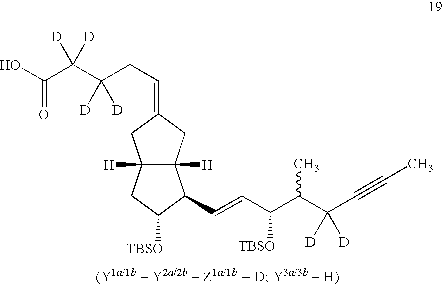

Two round-bottom flasks were flame-dried under nitrogen. One flask was charged with 17b (718 mg, 1.61 mmol, see Example 6 for preparation) and then flushed with nitrogen. The compound was suspended in benzene to form a slurry, the flask was fitted onto a rotary evaporator, and the benzene was removed in vacuo. (Note that for this procedure, gas-tight plastic syringes were used to add all liquid reagents.) A nitrogen balloon was attached to the rotary evaporator and the flask was back-filled with nitrogen. The azeotrope procedure described above was repeated twice and then the flask was charged with a stir bar, placed in a vacuum desiccator containing Drierite and subjected to high vacuum for approximately 30 min. During this time, the second round-bottom flask was charged with Compound 18 (Z^{1a}=Z^{1b}=D)(160 mg, 0.316 mmol, see Example for preparation) and then flushed with nitrogen. Compound 18 (Z^{1a}=Z^{1b}=D) was subjected to the azeotrope procedure described above three times, and then the flask was charged with a stir bar, placed in the vacuum desiccator and subjected to high vacuum for approximately 45 min. During this time the flask containing 17b was placed under nitrogen and dry THF (8.91 mL from a new bottle, ≧99.9%, inhibitor-free, Sigma-Aldrich) was added. To the resulting suspension was added t-BuOK (3.48 mL, 3.48 mmol, 1M solution in THF from a recently-opened Sigma-Aldrich bottle, stored in a desiccator when not in use). The resulting red, cloudy mixture was stirred rapidly for 30 min. The flask containing Compound 18 (Z^{1a}=Z^{1b}=D) was placed under nitrogen. THF (3.16 mL) was added, and the resulting solution was added quickly via cannula to the solution containing 17b. This was followed with a THF rinse (1.61 mL). After stirring overnight, the reaction was quenched with 50% citric acid in D₂O and diluted with EtOAc. The organic layer was washed twice with 50% citric acid in D₂O. The combined aqueous solutions were washed twice with EtOAc. The combined organic solutions were dried (Na₂SO₄), filtered and concentrated. Purification on an ISCO instrument (40 g SiO₂, 12.5% EtOAc in heptanes) afforded 188 mg (quant.) of 5. ¹H NMR (300 MHz, C₆D₆) Of mixture of diastereomers (only chemical shifts of major diastereomer are reported): δ 5.65 (m, 1H), 5.55 (m, 1H), 5.16 (br s, 1H), 4.20 (td, J=55.0, 4.9, 1H), 3.73 (q, J=8.2, 1H), 2.50-2.31 (m, 1H), 2.31-2.05 (m, 6H), 2.05-1.95 (m, 2H), 1.94-1.82 (m, 3H), 1.65 (s, 2H), 1.62-1.58 (m, 3H), 1.39-1.24 (m, 2H), 1.22-1.12 (m, 3H), 1.04 (s, 9H), 1.01 (s, 9H), 0.17 (s, 3H), 0.16 (s, 3H), 0.12 (s, 3H), 0.10 (s, 3H). MS (M+H): 594.7.

### Step 2. (E)-2,2,3,3-d₄-5-((3aS,4R,5R,6aS)-4-((R,E)-5,5-d₂-3-Hydroxy-4-methyloct-1-en-6-ynyl)-5-hydroxyhexahydropentalen-2(1H)-ylidene)pentanoic acid, (Compound 105)

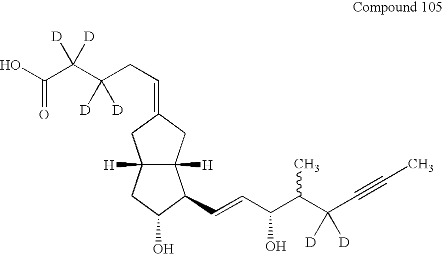

A 50-mL round-bottomed flask was charged with Compound 19 (Y^{1a}═Y^{1b}═Y^{2a}═Y^{2b}=Z^{1a}=Z^{1b}=D; Y^{3a}═Y^{3b}═H) (213 mg, 0.358 mmol) anhydrous THF (3.6 mL), and tetrabutylammonium fluoride (TBAF) (1.0 M solution in THF, 2.15 mL, 2.15 mmol). The reaction mixture was stirred for 48 hours with monitoring by TLC analysis. The reaction mixture was diluted with ethyl acetate (5 mL), treated with D₂O (5 mL), and adjusted to pH=3 with 5% DCl in D₂O solution. The aqueous layer was extracted with ethyl acetate (3×25 mL) and the combined organic extracts were concentrated to an oil. The oil was purified by column chromatography on silica gel (10 cm×1.5 cm; eluent 5% MeOH in CH₂Cl₂) to deliver Compound 105 as a yellow oil (96 mg, 73% yield) and a portion of Compound 105 as a mixture of EIZ isomers (86 mg). The oil containing the desired isomer was dissolved in ethyl acetate (15 mL) and washed with 10% DCl in D₂O solution (3×3 mL) to deliver Compound 105 as a pale yellow oil (13 mg, 10%): HPLC (Column: Waters SunFire Prep Silica, 5 μm, 4.6×250 mm column; Mobile Phase: 96:4 hexanes/i-PrOH (1.5 mL/min); Wavelength: 254 nm): retention time: 12.66/13.45 minutes (Z) and 17.79/18.92 minutes (E); 82.9% purity (E). ¹H NMR (300 MHz, C₆D₆) of mixture of diastereomers (only chemical shifts of major diastereomer are reported): δ 5.87-5.77 (m, 1H), 5.69-5.57 (m, 1H), 5.32 (br s, 1H), 4.30-4.19 (m, 1H), 3.87-3.79 (m, 1H), 2.50-2.02 (m, 10H), 1.74-1.72 (m, 3H), 1.47-1.23 (m, 7H). MS (M−H): 365.2.

The oil containing a mixture of E/Z isomers was dissolved in ethyl acetate (15 mL) and washed with 10% DCl in D₂O solution (3×3 mL). The organic layer was concentrated to an oil and purified by column chromatography on silica gel (10 cm×1.5 cm; eluent 5% MeOH in CH₂Cl₂). Purification delivered only mixed fractions, which were collected and concentrated to an oil. The oil was purified by column chromatography on silica gel (10 cm×1.5 cm; eluent 3% MeOH in CH₂Cl₂) to deliver Compound 102 as an oil (1 mg, 0.01%). Due to the small quantity of material, spectral data was not acquired for this material.

### Example 9

### Synthesis of (4-Carboxy-2,2,3,3-d₄-butyl)triphenylphosphonium bromide (17c) (Y^{1a/1b}═H; Y^{2a/2b}═Y^{3a/3b}=D)

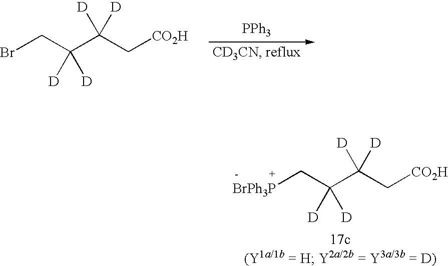

To a solution of commercially available 5-bromopentanoic acid (1.87 g, 10.1 mmol, 99.7% D) in CD₃CN (20 mL), was added triphenylphosphine (2.65 g, 10.1 mmol, 1.0 equiv). The mixture was heated to reflux for a period of 15 hours then cooled to ambient temperature. The cooled solution was concentrated to one-half volume then diluted with Et₂O until a cloudy mixture was obtained. Crystallization was initiated by scratching the inside wall of the flask which resulted in the formation of a white precipitate. The mixture was filtered and the material washed with Et₂O. The pure material was lyophilized to remove trace solvents and afforded a white solid (3.62 g, 80%) of 17c. MS: m/z 367.1 [M⁺].

### Example 10

### Synthesis of (E)-3,3,4,4-d₄-5-((3aS,4R,5R,6aS)-4-((R,E)-5,5-d₂-3-hydroxy-4-methyloct-1-en-6-ynyl)-5-hydroxyhexahydropentalen-2(1H)-ylidene)pentanoic acid Compound 114)

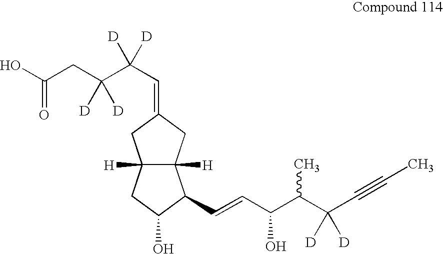

To a suspension of 17c (881 mg, 1.97 mmol, see Example 9 for preparation) in anhydrous THF (11 mL) was added KOt-Bu (1.0 M in THF, 4.34 mL, 4.34 mmol). The resultant orange solution was stirred at ambient temperature for 30 minutes followed by the addition of a solution of (18) (Z^{1a}=Z^{1b}=D) (200 mg, 0.395 mmol, see Example 5 for preparation) in THF (4 mL). The mixture was stirred at ambient temperature for 5 hours, then was quenched by the addition of saturated aqueous NH₄Cl solution (20 mL). The aqueous layer was extracted with EtOAc (3×25 mL) and the combined organic extracts were concentrated to an oil, which was purified by column chromatography on silica gel (10×3 cm; eluent 7:1 heptanes/EtOAc). The fractions containing product were separated into 2 portions and were concentrated to deliver 2 batches of product: 1) the product as a mixture of diastereomers (12 mg, 5% yield); and 2) the desired, more-polar compound with minor contamination by the undesired diastereomer (97 mg, 41% yield).

To a solution of the TBS ether of Compound 114 (97 mg, 0.163 mmol, batch 2 above) in anhydrous THF (1.6 mL) was added TBAF (1.0 M solution in THF, 978 μL, 0.978 mmol). The reaction mixture was stirred for 34 hours at ambient temperature, then the reaction was quenched by the addition of saturated aqueous NH₄Cl solution (5 mL). The mixture was diluted with water (5 mL), and was extracted with EtOAc (3×25 mL). The combined organic extracts were concentrated to an oil, and the resulting oil was purified by column chromatography on silica gel (10 cm×1.5 cm; eluent 5% MeOH in CH₂Cl₂) to deliver the desired isomer as a pale yellow oil (37 mg, 63% yield) and a portion of product as a mixture of diastereomers (16 mg, 27% yield).

A solution of the pure product above (37 mg) in EtOAc (10 mL) was washed with water (10 mL) and the pH of the aqueous layer was tested and found to be pH=6. The solution was washed with 5% aq HCl (0.5 mL) to adjust the pH of the aqueous layer to pH=3-4. The organic layer was removed and washed with two additional portions of the water (10 mL) and 5% aq HCl (0.5 mL) mixture. The organic layer was concentrated to deliver 12 mg of Compound 114 as an oil (12 mg, 20% yield). HPLC (Column: Waters SunFire Prep Silica, 5 μm, 4.6×250 mm column; Mobile Phase: 96:4 hexanes/i-PrOH (1.5 mL/min); Wavelength: 254 nm): retention time: 13.38/14.38 minutes (Z) and 18.42/19.61 minutes (E); 85.6% purity (E). ¹H NMR (300 MHz, C₆D₆) of mixture of diastereomers (only chemical shifts of major diastereomer are reported): δ 5.76-5.66 (m, 1H), 5.58-5.46 (m, 1H), 5.20 (br s, 1H), 4.19-4.08 (m, 1H), 3.76-3.68 (m, 1H), 2.38-1.89 (m, 10H), 1.62-1.60 (m, 3H), 1.35-0.92 (m, 7H). MS (M−H): 365.4.

### Example 11

### Synthesis of (E)-3,3,4,4-d₄-5-((3aS,4R,5R,6aS)-4-((R,E)-3-Hydroxy-4-methyloct-1-en-6-ynyl)-5-hydroxyhexahydropentalen-2(1H)-ylidene)pentanoic acid Compound 111)

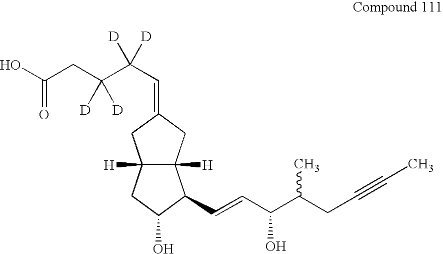

To a suspension of 17c (886 mg, 1.98 mmol, see Example 9 for preparation) in THF (11 mL) was added KOt-Bu (1.0 M solution in THF, 4.36 mL, 4.36 mmol). The mixture was stirred for 30 minutes at ambient temperature then was treated with (18) (Z^{1a}=Z^{1b}=H) (200 mg, 0.396 mmol, see Example 4 for preparation) in THF (4 mL). After 4 hours, the mixture was treated with NH₄Cl solution (15 mL) and adjusted to pH=4 with 10% aqueous citric acid solution. The aqueous layer was extracted with EtOAc (3×50 mL) and the combined organic extracts were concentrated to an oil and purified by column chromatography on silica gel (15 cm×1.5 cm; eluent 7:1 heptanes/EtOAc) to deliver the product as a yellow oil (137 mg, 59% yield).

A solution of the TBS ether of Compound 111 (137 mg, 0.231 mmol) in THF (2.5 mL was treated with TBAF (1.0 M solution in THF, 1.4 mL, 1.39 mmol) and stirred for 48 hours. The reaction mixture was diluted with EtOAc (5 mL), treated with saturated aqueous NH₄Cl solution (5 mL) and adjusted to pH=3 with 5% HCl. The aqueous layer was extracted with EtOAc (3×25 mL), and the combined organic extracts were washed with brine (50 mL) and concentrated to an oil. Purification of the resulting oil by column chromatography on silica gel (10×1.5 cm; eluent 5% MeOH in CH₂Cl₂) afforded Compound 111 as a pale yellow oil (16 mg, 20% yield). HPLC (Column: Waters SunFire Prep Silica, 5 μm, 4.6×250 mm column; Mobile Phase: 96:4 hexanes/i-PrOH (1.5 mL/min); Wavelength: 254 nm): retention time: 14.75/15.72 minutes (Z) and 20.74/21.99 minutes (E); 86.1% purity (E). ¹H NMR (300 MHz, C₆D₆) of mixture of diastereomers (only chemical shifts of major diastereomer are reported): δ 5.76-5.62 (m, 1H), 5.59-5.41 (m, 1H), 5.16 (br s, 1H), 4.38-4.05 (m, 1H), 3.70-3.65 (m, 1H), 2.41-1.90 (m, 12H), 1.58-1.56 (m, 3H), 1.30-1.08 (m, 6H), 0.96-0.82 (m, 1H). MS (M−H): 363.4.

### Evaluation of Metabolic Stability

Certain in vitro liver metabolism studies have been described previously in the following references, each of which is incorporated herein in their entirety: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al., Pharmacol Ther, 1997, 73:147; and Lave, T, et al., Pharm Res, 1997, 14:152.

Microsomal Assay: The metabolic stability of compounds of Formula I or II or III is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

Experimental Procedures: Human liver microsomes are obtained from a commercial source (e.g., XenoTech, LLC (Lenexa, Kans.)). The incubation mixtures are prepared as follows:

### Reaction Mixture Composition

| | | |
|---|---|---|
| Liver Microsomes | 0.5-2.0 | mg/mL |
| NADPH | 1 | mM |
| Potassium Phosphate, pH 7.4 | 100 | mM |
| Magnesium Chloride | 10 | mM |
| Test Compound | 0.1-1 | μM. |

Incubation of Test Compounds with Liver Microsomes: The reaction mixture, minus cofactors, is prepared. An aliquot of the reaction mixture (without cofactors) is incubated in a shaking water bath at 37° C. for 3 minutes. Another aliquot of the reaction mixture is prepared as the negative control. The test compound is added into both the reaction mixture and the negative control at a final concentration of 1 μM. An aliquot of the reaction mixture is prepared as a blank control, by the addition of plain organic solvent (not the test compound). The reaction is initiated by the addition of cofactors (not into the negative controls), and then is incubated in a shaking water bath at 37° C. Aliquots (200 μL) are withdrawn in triplicate at multiple time points (e.g., 0, 15, 30, 60, and 120 minutes) and are combined with 800 μL of ice-cold 50/50 acetonitrile/dH₂O to terminate the reaction. The positive controls, testosterone and propranolol, as well as iloprost, are each run simultaneously with the test compounds in separate reactions.

All samples are analyzed using LC-MS (or MS/MS). An LC-MRM-MS/MS method is used for metabolic stability. Also, Q1 full scan LC-MS methods are performed on the blank matrix and the test compound incubation samples. The Q1 scans serve as survey scans to identify any sample unique peaks that might represent the possible metabolites. The masses of these potential metabolites can be determined from the Q1 scans.

SUPERSOMES™ Assay. Various human cytochrome P450-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a compound of Formula I or II or III in 100 mM potassium phosphate buffer (pH 7.4) is incubated at 37° C. in triplicate. Positive controls contain 1 μM of iloprost instead of a compound of Formula I or II or III. Negative controls use Control Insect Cell Cytosol (insect cell microsomes that lack any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 50 μL of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of formula I: 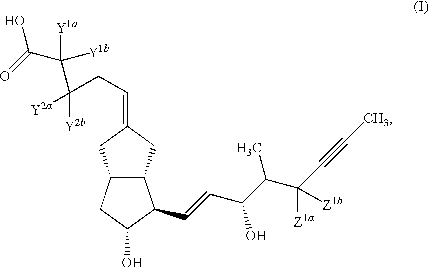
or a pharmaceutically acceptable salt thereof, wherein:
each Y is independently selected from hydrogen or deuterium;
each Z is independently selected from hydrogen, deuterium or fluorine; and
at least one Y or Z is deuterium.

2. The compound of claim 1, wherein Y^{1a} and Y^{1b} are the same.

3. The compound of claim 2, wherein Y^{1a} and Y^{1b} are simultaneously deuterium.

4. The compound of claim 1, wherein Y^{2a} and Y^{2b} are the same.

5. The compound of claim 4, wherein Y^{2a} and Y^{2b} are simultaneously deuterium.

6. The compound of claim 1, wherein Z^{1a} and Z^{1b} are the same.

7. The compound of claim 6, wherein Z^{1a} and Z^{1b} are simultaneously deuterium.

8. The compound of claim 1 selected from any one of the compounds set forth in the following table:

| Cmpd | Y^{1a} | Y^{1b} | Y^{2a} | Y^{2b} | Z^{1a} | Z^{1b} |
|---|---|---|---|---|---|---|
| 100 | D | D | H | H | H | H |
| 101 | H | H | D | D | H | H |
| 102 | D | D | D | D | H | H |
| 103 | D | D | H | H | D | D |
| 104 | H | H | D | D | D | D |
| 105 | D | D | D | D | D | D |
| 106 | D | D | H | H | F | F |
| 107 | H | H | D | D | F | F |
| 108 | D | D | D | D | F | F |
| 109 | H | H | H | H | D | D |

9. A compound of formula II: 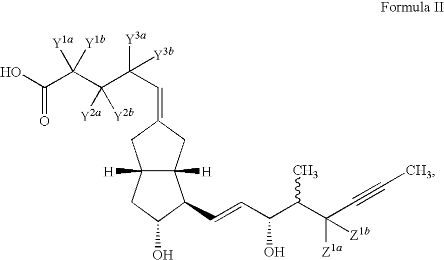
or a pharmaceutically acceptable salt thereof, wherein:
each Y is independently selected from hydrogen or deuterium;
each Z is independently selected from hydrogen and deuterium; and
at least one Y³ is deuterium.

10. The compound of claim 9, wherein Y^{3a} and Y^{3b} are simultaneously deuterium.

11. The compound of claim 9, wherein Y^{1a} and Y^{1b} are the same.

12. The compound of claim 11, wherein Y^{1a} and Y^{1b} are simultaneously deuterium.

13. The compound of claim 9, wherein Y^{2a} and Y^{2b} are the same.

14. The compound of claim 13, wherein Y^{2a} and Y^{2b} are simultaneously deuterium.

15. The compound of claim 9, wherein Z^{1a} and Z^{1b} are the same.

16. The compound of claim 15, wherein Z^{1a} and Z^{1b} are simultaneously deuterium.

17. The compound of claim 9, wherein the compound is selected from any one of the compounds set forth in the following table:

| Cmpd | Y^{1a} | Y^{1b} | Y^{2a} | Y^{2b} | Y^{3a} | Y^{3b} | Z^{1a} | Z^{1b} |
|---|---|---|---|---|---|---|---|---|
| 110 | D | D | H | H | D | D | H | H |
| 111 | H | H | D | D | D | D | H | H |
| 112 | D | D | D | D | D | D | H | H |
| 113 | D | D | H | H | D | D | D | D |
| 114 | H | H | D | D | D | D | D | D |
| 115 | D | D | D | D | D | D | D | D |

18. A compound of claim 1, wherein any atom not designated as deuterium is present at its natural isotopic abundance.

19. A compound selected from: 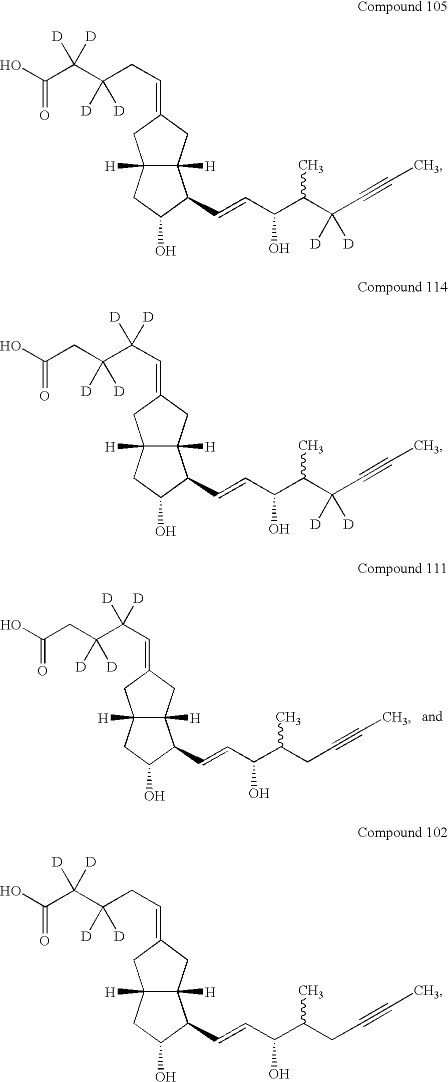
or a pharmaceutically acceptable salt of any of the foregoing compounds.

20. A pyrogen-free composition comprising an effective amount of a compound according to claim 1, and a pharmaceutically acceptable carrier.

21. The composition according to claim 20, wherein said composition is an inhalable microparticle formulation.

22. The composition according to claim 20, wherein said composition is an oral formulation.

23. The composition according to claim 20, additionally comprising a second therapeutic agent.

24. The composition according to claim 23, wherein said second therapeutic agent is an agent useful in the treatment or prevention of a disease or condition selected from pulmonary arterial hypertension, Raynaud's Phenomenon secondary to systemic sclerosis, contrast-mediated nephropathy, and lung cancer.

25. The composition according to claim 24, wherein said second therapeutic agent co-formulated with a compound of this invention is an agent useful in the treatment of pulmonary arterial hypertension.

26. The composition according to claim 25, wherein said second therapeutic agent is selected from a phosphodiesterase V inhibitor or an endothlin-1 antagonist.

27. The composition according to claim 26, wherein said second therapeutic agent is sildenafil.

28. The composition according to claim 26, wherein said second therapeutic agent is bosentan.

29. A method of modulating the activity of a prostacyclin receptor in a cell comprising contacting the cell with a compound of claim 1.

30. A method of treating a patient suffering from or susceptible to a disease or condition selected from pulmonary arterial hypertension, Raynaud's phenomenon secondary to systemic sclerosis, contrast-mediated nephropathy, and lung cancer comprising the step of administering to the patient in need thereof a composition of claim 20.

31. The method according to claim 30, wherein said disease is pulmonary arterial hypertension.

32. The method according to claim 31, comprising the additional step of administering to the patient in need thereof a second therapeutic agent selected from a phosphodiesterase V inhibitor, and an endothelin-1 antagonist.

33. The method according to claim 32, wherein said second therapeutic agent is sildenafil.

34. The method according to claim 32, wherein said second therapeutic agent is Bosentan.

35. A method of treating a patient suffering from or susceptible to a disease or a condition comprising the step of co-administering to the patient in need thereof a composition of claim 20 and a second therapeutic agent, wherein: the disease or condition is:
a. erectile dysfunction and the second therapeutic agent is a 15-hydroxyprostaglndindehydrogenase inhibitor;
b. a thrombotic condition, and the second therapeutic agent is a betaine;
c. selected from angina, high blood pressure, pulmonary hypertension, congestive heart failure, chronic obstructive pulmonary disease (COPD), pulmonary heart disease, right ventricular failure, atherosclerosis, permeability conditions of reduced cardiovascular patency, peripheral vascular illnesses, cerebral apoplexy, bronchitis, allergic asthma, chronic asthma, allergic rhinitis, glaucoma, irritable bowel syndrome, tumors, kidney failure, cirrhosis of the liver male sexual problems and female sexual problems, and the second therapeutic agent is a phosphodiesterase V inhibitor;
d. an inflammation-related cardiovascular, and the second therapeutic agent is a COX-1 or COX-2 inhibitor;
e. insufficient hair thickness, and the second therapeutic agent is a 15-hydroxyprostaglndindehydrogenase inhibitor;
f. multiple sclerosis, and the second therapeutic agent is a cannabidiol derivative;
g. bacterial infection, and the second therapeutic agent is an α1-antitrypsin or a serine protease inhibitor;
h. lung proliferative vascular disorder, and the second therapeutic agent is a HMG-CoA reductase inhibitor;
i. pulmonary hypertension and the second therapeutic agent is thalidomide or a phosphodiesterase IV inhibitor;
j. selected from hypertension, complications in diabetes and metabolic syndrome, and the second therapeutic agent is a blood pressure lowering agent; or
k. pulmonary arterial hypertension, and the second therapeutic agent is selected from an endothelin receptor antagonist, a phosphodiesterase inhibitor and a calcium channel blocker.
